(19) United States
(12) Patent Application Publication
(10) Pub. No.: US 2024/0117288 A1
(43) Pub. Date: Apr. 11, 2024

(54) CULTURE DEVICES

(71) Applicant: SHANGHAI RUIYU BIOTECH CO., LTD., Shanghai (CN)

(72) Inventor: Xuan WANG, Shanghai (CN)

(73) Assignee: SHANGHAI RUIYU BIOTECH CO., LTD., Shanghai (CN)

(21) Appl. No.: 18/393,762

(22) Filed: Dec. 22, 2023

Related U.S. Application Data
(63) Continuation of application No. PCT/CN2021/119589, filed on Sep. 22, 2021.

(30) Foreign Application Priority Data
Jun. 25, 2021 (CN) 202110712508.4
Jun. 25, 2021 (CN) 202110726570.9

(51) Int. Cl. C12M 1/12; C12M 1/00

(52) U.S. Cl. CPC C12M 25/04; C12M 23/38

(57) ABSTRACT

One or more embodiments of the present disclosure disclose a culture device. The culture device may include a chamber layer including at least one culture chamber for accommodating a culture; a sample introduction channel in flow communication with the at least one culture chamber, the sample introduction channel being configured to guide the culture into or out of the at least one culture chamber; and a culture fluid channel in flow communication with the at least one culture chamber, the culture fluid channel being configured to guide a culture fluid into or out of the at least one culture chamber.

### CROSS REFERENCE TO RELATED APPLICATIONS

This application is a continuation of International application No. PCT/CN2021/119589 filed Sep. 22, 2021, which claims priority to Chinese application No. 202110712508.4, filed on Jun. 25, 2021, and Chinese application No. 202110726570.9, filed on Jun. 25, 2021, the entire contents of each of which are incorporated herein by reference.

### TECHNICAL FIELD

The present disclosure relates to the technical field of cell culture, and in particular, to a culture device.

### BACKGROUND

Culture cultivating may include adding a sample culture, adding a sample culture fluid, or updating or exchanging the sample culture fluid. In some situations, it may be necessary to add other substances to a culture chamber after the culture has been cultured. For example, in drug screening, a drug to be screened needs to be added to the culture chamber. Currently, the process of adding the sample culture, adding or updating the sample culture fluid or other substances usually needs to be performed well by well, which is inefficient. In addition, the culture may be affected when adding, exchanging, or updating the culture fluid or other substances. For example, the culture may be lost as the culture fluid is transported into and out of the culture chamber. Moreover, the culture chamber may be made of a dense material that is impermeable to any substance. This lack of permeability may hinder an exchange of substances inside and outside the culture chamber, which is not conducive to the automated cultivation of culture.

### SUMMARY

Some embodiments of the present disclosure may provide a culture device, comprising: a chamber layer including at least one culture chamber for accommodating a culture; a sample introduction channel in flow communication with the at least one culture chamber, the sample introduction channel being configured to guide the culture into or out of the at least one culture chamber; and a culture fluid channel in flow communication with the at least one culture chamber, the culture fluid channel being configured to guide a culture fluid into or out of the at least one culture chamber.

In some embodiments, at least one of the sample introduction channel or the culture fluid channel may be disposed independently relative to the chamber layer.

In some embodiments, the sample introduction channel may be disposed above the culture fluid channel.

In some embodiments, the sample introduction channel may include a sample introduction plate and a culture inlet and outlet channel, the culture inlet and the outlet channel being disposed on the sample introduction plate, and the culture inlet and outlet channel being in flow communication with the at least one culture chamber.

In some embodiments, the culture fluid channel may comprise a culture fluid inlet for inputting the culture fluid; a culture fluid outlet for discharging the culture fluid; and a culture fluid update channel configured to transport the culture fluid input via the culture fluid inlet to the at least one culture chamber.

In some embodiments, wherein the at least two culture chambers may include at least two sets of culture chambers, and each set of the at least two sets of culture chambers may include one or more culture chambers among the at least one culture chamber; and the culture fluid channel may include at least two culture fluid sub-channels, each of the at least two culture fluid sub-channels may correspond to one set of the at least two sets of culture chambers, and each of the at least two culture fluid sub-channels may include a culture fluid sub-inlet, a culture fluid sub-outlet, and a culture fluid update sub-channel.

In some embodiments, wherein the culture fluid outlet may have a first connection with the at least one culture chamber, the culture fluid update channel may have a second connection with the at least one culture chamber, and a cross-sectional dimension of at least one of the first connection and the second connection may be smaller than a dimension of a constituent unit of the culture.

In some embodiments, wherein the culture fluid outlet may have a first connection with the at least one culture chamber; the culture fluid update channel may have a second connection with the at least one culture chamber; and at least one of the first connection and the second connection may be provided with a porous membrane for retaining the culture.

In some embodiments, a pore size of the porous membrane may be no larger than 5 micrometers.

In some embodiments, the pore size of the porous membrane may be in a range of 50 micrometers to 4 millimeters.

In some embodiments, at least one of the culture fluid inlet, the culture fluid outlet, or the culture fluid update channel may be integrally disposed on a surface of the chamber layer or within the chamber layer.

In some embodiments, wherein the culture fluid outlet may be located above the culture fluid update channel.

In some embodiments, the chamber layer may comprise a culture plate, and the culture plate may be provided with at least one through well; the culture device may further comprise a sealing cover, wherein the sealing cover may cover a bottom surface of the culture plate and form the least one culture chamber with the at least one through well.

In some embodiments, wherein the sealing cover may have at least one concave structure; the at least one through well and the at least one concave structure may form the at least one culture chamber.

In some embodiments, the chamber layer may comprise a culture plate; at least one porous membrane disposed on the culture plate, wherein the at least one porous membrane may form the at least one culture chamber with the culture plate, and the at least one porous membrane may be attached to or configured to form a side wall of the at least one culture chamber.

In some embodiments, the chamber layer may further comprise a membrane of inert material disposed on an inner bottom wall of the at least one culture chamber.

In some embodiments, wherein the culture device may further comprise at least one rack disposed on the culture plate, and each porous membrane of the at least one porous membrane may be attached to a peripheral wall of one of the at least one rack.

In some embodiments, wherein the peripheral wall of the rack may be provided with a skeletonized structure, and the skeletonized structure may be configured to enable circulation of the culture fluid.

In some embodiments, wherein the at least one porous membrane may comprise at least one of a hollow fiber membrane, a tubular membrane, a ceramic membrane, or a polymer membrane.

In some embodiments, the chamber layer may comprise: a culture plate; at least one through well disposed on the culture plate; and a porous membrane disposed at a bottom end of each of the at least one through well, and the each through well may form the culture chamber with the porous membrane disposed at the bottom end of the through well.

In some embodiments, wherein the culture fluid channel may comprise a culture fluid accommodation chamber with an open end, and the culture fluid accommodation chamber may be disposed below the culture plate.

In some embodiments, wherein the at least one culture chamber may comprise at least two sets of culture chambers, and each set of the at least two sets of culture chambers may comprise one or more culture chambers of the at least one culture chamber; and the culture fluid accommodation chamber may comprise at least two culture fluid accommodation sub-channels, each of the at least two culture fluid accommodation sub-channels may correspond to one set of the at least two sets of culture chambers, and each of the at least two culture fluid accommodation sub-channels may comprise an accommodation chamber inlet and an accommodation chamber outlet disposed independently.

In some embodiments, wherein the at least two culture fluid accommodation sub-chambers may form a grid structure or a side-by-side channel structure.

In some embodiments, wherein an inert material may be attached to a surface of the porous membrane.

In some embodiments, wherein the porous membrane may form a concave structure.

In some embodiments, wherein a pore size of the porous membrane may be in a range of 0.1 nanometers to 1 nanometer.

In some embodiments, the pore size of the porous membrane may be in a range of 1 nanometer to 100 nanometers.

In some embodiments, the pore size of the porous membrane may be in a range of 5 nanometers to 1 micron.

In some embodiments, the pore size of the porous membrane may be in a range of 100 nanometers to 10 micrometers.

In some embodiments, the pore size of the porous membrane may be in a range of 10 micrometers to 1 centimeter.

In some embodiments, wherein a ratio of a length to a diameter of the at least one culture chamber may be in a range of 1 to 20.

In some embodiments, wherein a diameter of an inscribed circle of the at least one culture chamber may be no less than 5 micrometers.

In some embodiments, wherein a diameter of an inscribed circle of the at least one culture chamber may be in a range of 5 micrometers to 10 micrometers.

In some embodiments, wherein a diameter of an inscribed circle of the at least one culture chamber may be in a range of 10 micrometers to 1000 micrometers.

In some embodiments, wherein the diameter of an inscribed circle of the at least one culture chamber may be in a range of 100 micrometers to 5 centimeters.

In some embodiments, wherein a diameter of an inscribed circle of the at least one culture chamber may be in a range of 1 centimeter to 1 meter.

In some embodiments, wherein a shape of the at least one culture chamber may comprise at least one of a cylindrical chamber or a prismatic chamber.

### BRIEF DESCRIPTION OF THE DRAWINGS

The present disclosure is further illustrated in terms of exemplary embodiments. These exemplary embodiments are described in detail with reference to the drawings. These embodiments are not limiting, and in these embodiments, the same numbering denotes the same structure, wherein:

FIG. 1 is a block diagram illustrating an exemplary culture device according to some embodiments of the present disclosure;

FIG. 2 is a schematic diagram illustrating an exemplary explosion structure of a culture device according to some embodiments of the present disclosure;

FIG. 3 is a schematic diagram illustrating an exemplary structure of the culture device illustrated in FIG. 2 after being assembled;

FIG. 4 is a schematic diagram illustrating an exemplary top view of a culture inlet and outlet channel and a chamber layer according to some embodiments of the present disclosure;

FIG. 5 is a schematic diagram illustrating an exemplary culture device according to some embodiments of the present disclosure;

FIG. 6 is a schematic diagram illustrating an exemplary chamber layer and an exemplary culture fluid channel according to some embodiments of the present disclosure;

FIG. 7 is a schematic diagram illustrating an exemplary bottom view of the chamber layer and the culture fluid channel illustrated in FIG. 6;

FIG. 8 is a schematic diagram illustrating an exemplary chamber layer and an exemplary culture fluid channel disposed on a top surface of the chamber layer according to some embodiments of the present disclosure;

FIG. 9 is a schematic diagram illustrating an exemplary chamber layer and an exemplary culture fluid channel disposed on a bottom surface of the chamber layer according to some embodiments of the present disclosure;

FIG. 10 is a schematic diagram illustrating an exemplary chamber layer and an exemplary culture fluid channel disposed inside the chamber layer according to some embodiments of the present disclosure;

FIG. 11 is a schematic diagram illustrating an exemplary cross-sectional structure of the chamber layer illustrated in FIG. 10;

FIG. 12 is a schematic diagram illustrating an exemplary chamber layer according to some embodiments of the present disclosure;

FIG. 13 is a schematic diagram illustrating an exemplary cross-sectional structure of the chamber layer illustrated in FIG. 12;

FIG. 14 is a schematic diagram illustrating an exemplary lower sealing cover according to some embodiments of the present disclosure;

FIG. 15 is a schematic diagram illustrating an exemplary side view of the lower sealing cover illustrated in FIG. 14;

FIG. 16 is a schematic diagram illustrating an exemplary structure of the chamber layer according to some embodiments of the present disclosure;

FIG. 17 is a schematic diagram illustrating an exemplary chamber layer according to some embodiments of the present disclosure;

FIG. 18 is a schematic diagram illustrating an exemplary chamber layer according to some embodiments of the present disclosure;

FIG. 19 is a schematic diagram illustrating an exemplary porous membrane assembled with a culture plate according to some embodiments of the present disclosure;

FIG. 20 is a schematic diagram illustrating an exemplary rack according to some embodiments of the present disclosure;

FIG. 21 is a schematic diagram illustrating an exemplary rack illustrated in FIG. 20 after being assembled with a porous membrane;

FIG. 22 is a schematic diagram illustrating another exemplary rack according to some embodiments of the present disclosure;

FIG. 23 is a schematic diagram illustrating an exemplary assembled culture device according to some embodiments of the present disclosure;

FIG. 24 is a schematic diagram illustrating an exemplary explosion structure of a culture device according to some embodiments of the present disclosure;

FIG. 25 is a schematic diagram illustrating an exemplary three-dimensional structure of a chamber layer according to some embodiments of the present disclosure;

FIG. 26 is a schematic diagram illustrating an exemplary side view of a chamber layer according to some embodiments of the present disclosure;

FIG. 27 is a schematic diagram illustrating an exemplary cross-sectional view of the chamber layer along a direction A-A illustrated in FIG. 26;

FIG. 28 is a schematic diagram illustrating an exemplary culture fluid accommodation chamber comprising a plurality of culture fluid sub-accommodation chambers according to some embodiments of the present disclosure;

FIG. 29 is a schematic diagram illustrating an exemplary culture fluid accommodation chamber comprising a plurality of culture fluid sub-accommodation chambers according to some embodiments of the present disclosure; and

FIG. 30 is a schematic diagram illustrating an exemplary culture fluid accommodation chamber comprising a plurality of culture fluid sub-accommodation chambers according to some embodiments of the present disclosure.

### DETAILED DESCRIPTION

In order to more clearly illustrate the technical solutions of the embodiments of the present disclosure, the accompanying drawings required to be used in the description of the embodiments are briefly described below. Obviously, the accompanying drawings in the following description are only some examples or embodiments of the present disclosure, and it is possible for a person of ordinary skill in the art to apply the present application to other similar scenarios based on the accompanying drawings without creative labor. Unless obviously obtained from the context or the context illustrates otherwise, the same numeral in the drawings refers to the same structure or operation.

As used in the disclosure and the appended claims, the singular forms “a,” “an,” and “the” include plural referents unless the content clearly dictates otherwise. In general, the terms “comprise,” “comprises,” and/or “comprising,” “include,” “includes,” and/or “including,” merely prompt to include steps and elements that have been clearly identified, and these steps and elements do not constitute an exclusive listing. The methods or devices may also include other steps or elements. The term “based on” is “based at least in part on.” The term “one embodiment” means “at least one embodiment;” the term “another embodiment” means “at least one other embodiment.” Related definitions of other terms will be given in the description below.

In some embodiments, culture cultivation refers to a method for simulating a specific environment in a culture chamber to allow a culture (e.g., a cultured cell) to survive, grow, multiply, and maintain major structures and functions. The specific environment includes at least one of an environment in a human body or in an animal body, an aseptic environment, an environment with a specific temperature, an environment with a specific acidity or alkalinity, an environment that conforms to a certain nutrient condition, or the like. In some embodiments, the culture (e.g., a sample culture) may be placed in the culture chamber and a culture fluid supplying nutrients may be added to the culture chamber, then the culture may be able to propagate and grow by absorbing nutrients from the culture fluid.

The culture may refer to an object that is cultivated. In some embodiments, the culture may comprise a sample cell for cell culture, which may also be referred to as a cultured cell. In some embodiments, the culture may be mixed with a matrix gel. In some embodiments, the culture may comprise cells mixed with the matrix gel. In some embodiments, the culture may be a single cell mixed with the matrix gel or a plurality of cells mixed with the matrix gel. In some embodiments, the plurality of cells may be a plurality of dispersed cells or may be a cell cluster. In some embodiments, the cell cluster may include a cell ball formed by a plurality of cells mixed, i.e., a mass formed by a plurality of cells aggregated together. In some embodiments, the culture may comprise three-dimensional cells mixed with the matrix gel. The three-dimensional cell refers to a product of a three-dimensional cell culture process, e.g., an organoid. For example, the organoid may include a brain organoid, a colon organoid, a liver organoid, a tumor organoid, a stomach organoid, or the like.

The culture fluid is a substance that provides nutrients necessary for the growth and reproduction of the culture, and the culture fluid may be a combination of different nutrients. During the process of cultivation, it is necessary to add a sample culture and add or update the culture fluid. The efficiency of adding the sample culture and adding or updating the culture fluid directly affects the culture efficiency. In some cases, the culture may be divided into sets and form control sets for study. For example, in a drug screening process, the effect and mechanism of a drug may be investigated by analyzing the response of cells to drugs of different types and concentrations. In practice, cells and the culture fluid may be added to a high-through culture plate (e.g., a 96-well plate, a 384-well plate, etc.) well by well. Further, the culture fluid is removed from the culture chamber and drugs to be screened of different concentrations and/or compositions are added to different control sets well by well (i.e., a fluid exchange operation). The whole process may involve the well-by-well addition of cells and culture fluid, the change of the culture fluid, the well-by-well addition of drugs, or the like. When a large count of cells is required to be cultivated, this process is inefficient and may fail to meet an application requirement. In some embodiments, auto-sampling devices such as robotic arms, mechanical guides, and rows of guns are used to add samples into a plurality of wells at the same time. On the one hand, it is costly to produce or purchase auto-sampling devices. On the other hand, during the process of removing the culture from the culture chamber, some of the culture may be aspirated, resulting in the culture being discarded. In such a case, how to efficiently add the sample culture and the sample culture fluid, and how to efficiently update the culture without affecting the culture chamber have become urgent problems.

For the foregoing reasons, some embodiments of the present disclosure provide a culture device whose sample introduction channel for the culture is independent of a culture fluid channel. An operator may add a sample culture into the culture chamber through the sample introduction channel, and add or update the culture fluid or other substances through the culture fluid channel. As used herein, updating the culture fluid refers to guiding at least a portion of the culture fluid into or out of the culture chamber, for example, guiding a culture fluid A out of the culture chamber and then guiding a culture fluid B (or other substances) into the culture chamber such that the culture fluid A is replaced by culture fluid B. Other substances in the present disclosure refer to substances other than the culture and the culture fluid, for example, the drugs to be screened, etc. In addition, since the culture, the culture fluid, or the other substances are added through different channels, when a subsequent study such as a drug screening is performed, the culture fluid may be guided out through a separate channel, and a drug to be screened may also be added through a separate channel, thereby avoiding any impact on the culture (e.g., resulting in a loss of the culture). In some embodiments, the sample introduction channel may be in flow communication with a plurality of culture chambers for adding the sample culture into a plurality of culture chambers at the same time. The culture fluid channel may also be in flow communication with the plurality of culture chambers for adding the sample culture fluid into the plurality of culture chambers and/or updating the sample culture fluid. In such cases, a cumbersome procedure of adding samples and changing fluids well by well may be avoided, which improves the cultivation efficiency of the culture. In some embodiments, the culture chamber may be divided into a plurality of sets of culture chambers, and each set of culture chambers may be provided with an independent c culture fluid channel, thereby realizing highly efficient cultivation by sets.

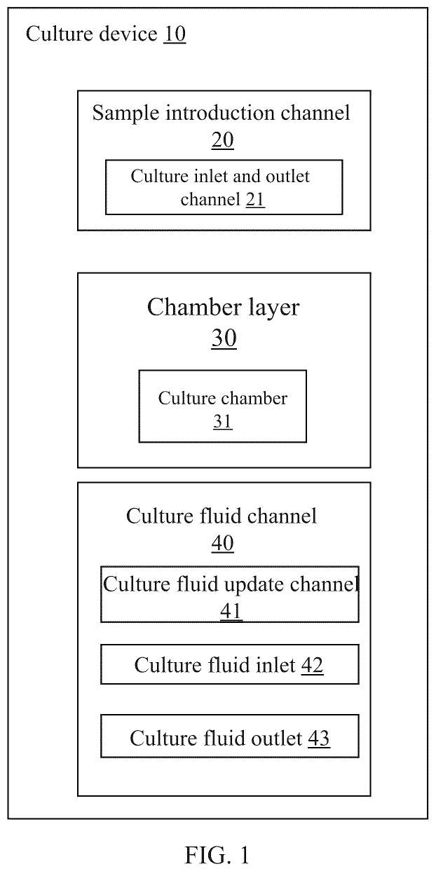

FIG. 1 is a block diagram illustrating an exemplary culture device according to some embodiments of the present disclosure. As shown in FIG. 1, a culture device 10 may include a sample introduction channel 20, a chamber layer 30, and a culture fluid channel 40.

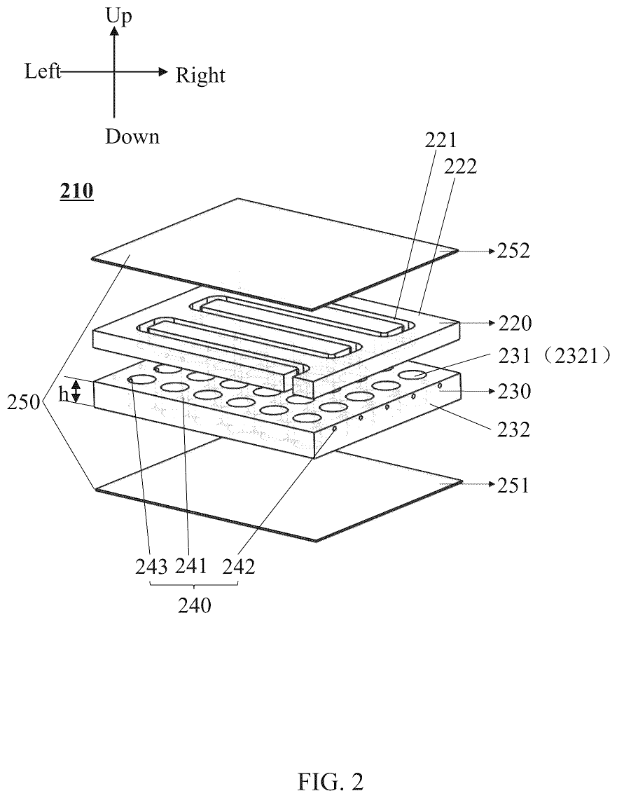

The chamber layer 30 may be used to accommodate a culture. In some embodiments, the chamber layer 30 may include at least one culture chamber 31 for accommodating the culture. A displacement of the culture chamber 31 may be in various forms. For example, the chamber layer 30 may comprise a culture plate (e.g., a culture plate 232 shown in FIG. 2) and at least one through well opened on the culture plate. The through well may form the culture chamber 31 with a sealing cover (not shown in FIG. 1) of the culture device 10. As another example, the culture chamber 31 may be formed by a blind well on the culture plate.

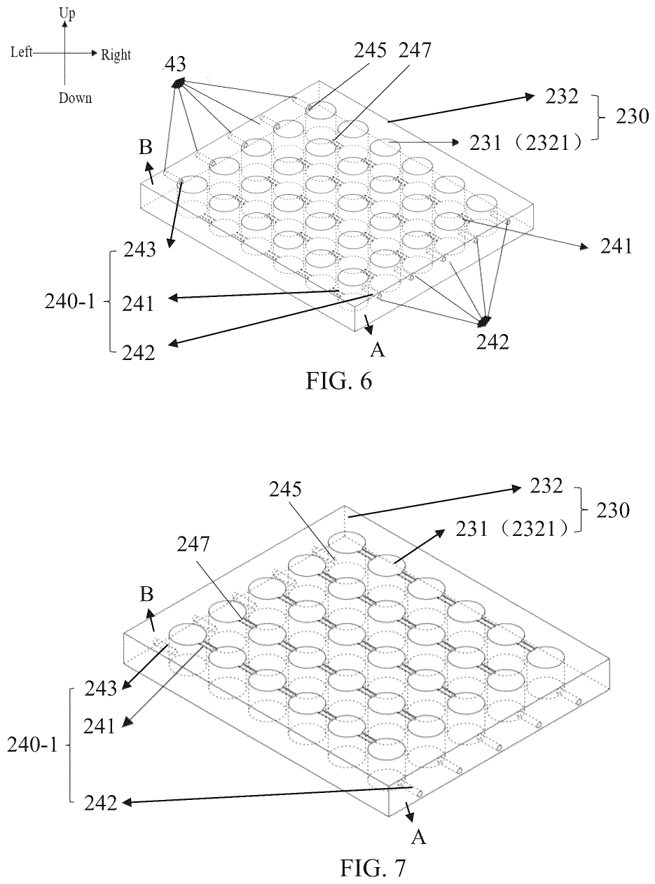

In some embodiments, a count of the at least one culture chamber 31 may be 1, 2, 3, or more. For example, in an embodiment shown in FIG. 6, the count of the culture chambers 231 is 30. The present disclosure does not limit the count of culture chambers 31, and it will be appreciated that the count of culture chambers 31 is related to a culture requirement. For example, the count of the culture chambers 31 is large when a large batch of culture is required to be cultivated.

In some embodiments, a bottom of the culture chamber 31 may be planar or non-planar (e.g., concave). As used herein, the bottom of the culture chamber 31 refers to a plane opposite to an opening of the culture chamber 31. In some embodiments, the bottom of the culture chamber 31 may be a downwardly concave surface. The bottom of the culture chamber 31 being a downwardly concave surface means that the bottom of the culture chamber 31 protrudes outwardly away from the interior of the culture chamber 31. In some embodiments, the shape of the bottom of the culture chamber 31 may be set based on a culture requirement. For example, when a two-dimensional culture is desired, the bottom of the culture chamber 31 may be planar to allow a culture (e.g., a cell) to attach to the bottom of the culture chamber, wherein the two-dimensional culture refers to that a culture grows on a surface of the culture chamber 31. As another example, when a three-dimensional culture is desired, the bottom of the culture chamber 31 may be set as a concave surface to accelerate the aggregation of the culture(s) (e.g., the cells) into a mass, wherein the three-dimensional culture refers to that the culture(s) grow within an inner space of the culture chamber 31.

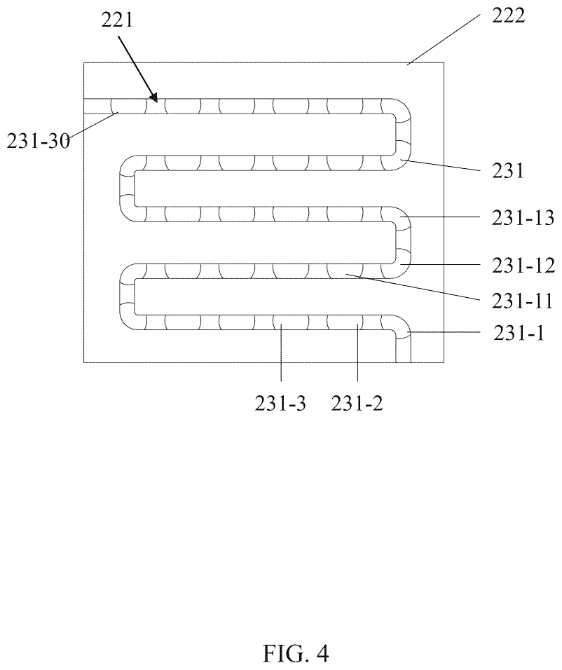

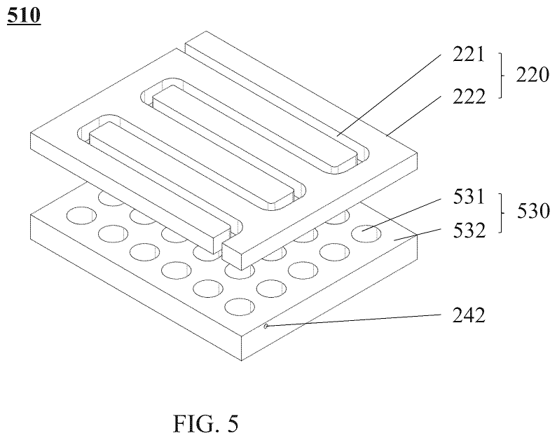

The sample introduction channel 20 may be in flow communication with the chamber layer 30 so that the culture is able to enter the chamber layer 30 through the sample introduction channel 20. In some embodiments, the sample introduction channel 20 may include a culture inlet and outlet channel 21. The culture may be added to the culture chamber 31 via the culture inlet and outlet channel 21 and/or guided out of the culture chamber 31 via the culture inlet and outlet channel 21. In some embodiments, the culture inlet and outlet channel 21 may include an inlet and an outlet. The culture may enter the culture inlet and outlet channel 21 from the inlet of the culture inlet and outlet channel 21, and then enter the culture chamber 31 which is connected to the culture inlet and outlet channel 21 through the culture inlet and outlet channel 21. Excess cultures may then be guided out via the outlet of the culture inlet and outlet channel 21. In some embodiments, a count and shape of the culture inlet and outlet channel 21 may be set based on an actual situation. More details about the culture inlet and outlet channel 21 can be found in the descriptions of other portions of the present disclosure (e.g., FIGS. 4 and 5), which are not repeated here.

In some embodiments, the culture fluid channel 40 may also be in flow communication with the chamber layer 30 for transporting the culture fluid or other substances into the chamber layer 30, or for updating the culture fluid or other substances.

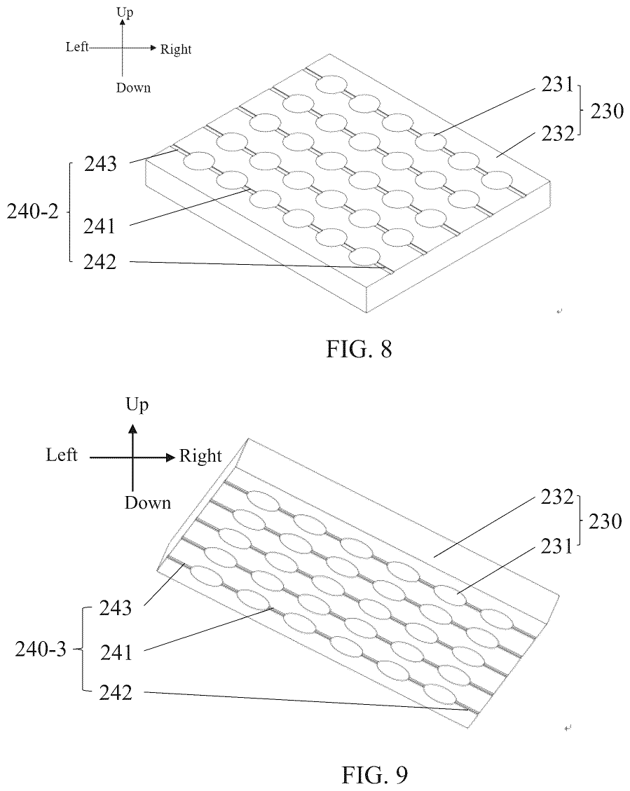

In some embodiments, the culture fluid channel 40 may include a culture fluid inlet 42 for inputting the culture fluid or other substances, a culture fluid outlet 43 for discharging the culture fluid or other substances, and a culture fluid update channel 41. The culture fluid update channel 41 may be used to transport the culture fluid or other substances input via the culture fluid inlet 42 into the culture chamber 31. For example, the culture fluid may be added via the culture fluid inlet 42 during a culture process, the culture fluid may sequentially enter different culture chambers 31 via the culture fluid update channel 41, and excess culture fluids or culture fluids that have been exchanged with culture fluids in the culture chambers 31 may be discharged through the culture fluid outlet 43. In some embodiments, when the culture fluid channel 40 is provided on the chamber layer 30, at least one of the culture fluid inlet 42, the culture fluid outlet 43, or the culture fluid update channel 41 may be integrally provided on a surface or interior of the chamber layer 30. More details regarding the culture fluid channel 40 can be found in the descriptions of other portions of the present disclosure (e.g., embodiments of FIGS. 8 to 15), which are not repeated herein.

The culture device 10 of the present disclosure is provided with the sample introduction channel 20 and the culture fluid channel 40, and the sample introduction channel 20 and the culture fluid channel 40 are independent of each other. In the culture process or a research process using the culture device 10 of the present disclosure, the sample introduction channel 20 may be utilized for adding cultures and the culture fluid channel 40 may be utilized for adding or updating the culture fluid and/or other substances. As a result, adding the cultures and adding or updating the culture fluid and/or other substances may be carried out independently, which may reduce the impact of adding or updating the culture fluid and/or other substances on the sample introduction channel 20 and/or the chamber layer 30. Additionally, in some embodiments, the sample introduction channel 20 and/or the culture fluid channel 40 may be in flow communication with a plurality of culture chambers 31. Such a design can avoid the cumbersome process of adding cultures and updating culture fluids well by well. In some embodiments, when the culture device 10 is applied in drug screening, operations of drug screening can be simplified and the efficiency of drug screening can be improved.

In some application scenarios, when the culture cultivation is complete, the culture fluid in the culture chamber 31 may be discharged through the culture fluid channel 40, and the culture fluid channel 40 may be used to add a drug to be screened to the culture chamber 31 or update the culture fluid in the culture chamber 31 with the drug to be screened. Since the culture fluid and/or other substances may be guided into or out of the culture chamber 31 through the culture fluid channel 40, and the culture cannot enter the culture fluid channel 40, a loss of culture while discharging the culture fluid and/or adding a drug may be avoided. In addition, there is no need to transfer the culture to other culture chambers when drug screening is performed using the culture device 10 of the present disclosure, which avoids waste of cultures, reduces the burden on an operator, and improves the drug screening efficiency.

In some embodiments, at least one of the sample introduction channel 20 or the culture fluid channel 40 may be disposed independently relative to the chamber layer 30. In the present disclosure, if the sample introduction channel 20 and the culture fluid channel 40 may be separated from each other, the two components are considered to be disposed independently. The two independently disposed components may be spliced or assembled, or they may not be spliced or assembled. For example, the sample introduction channel 20 and the chamber layer 30 may be two independently disposed components, and the culture fluid channel 40 is disposed on the chamber layer 30, i.e., the culture fluid channel 40 is integrated into the chamber layer 30. As another example, the culture fluid channel 40 and the chamber layer 30 may be two separate components, and the sample introduction channel 20 may be disposed on the chamber layer 30, i.e., the sample introduction channel 20 is integrated into the chamber layer 30. For a further example, the sample introduction channel 20 and the culture fluid channel 40 may both be disposed independently relative to the chamber layer 30. If the culture fluid channel 40 and/or the sample introduction channel 20 are provided in a same structure with the chamber layer 30 and are not separable, several channels for transporting the culture and/or the culture fluid are required to be opened in the chamber layer 30 while producing the chamber layer 30, which may make the production of the chamber layer 30 more difficult. Providing the culture fluid channel 40 and/or the sample introduction channel 20 independently of the chamber layer 30 may simplify the structure of the chamber layer 30, thereby reducing the production difficulty.

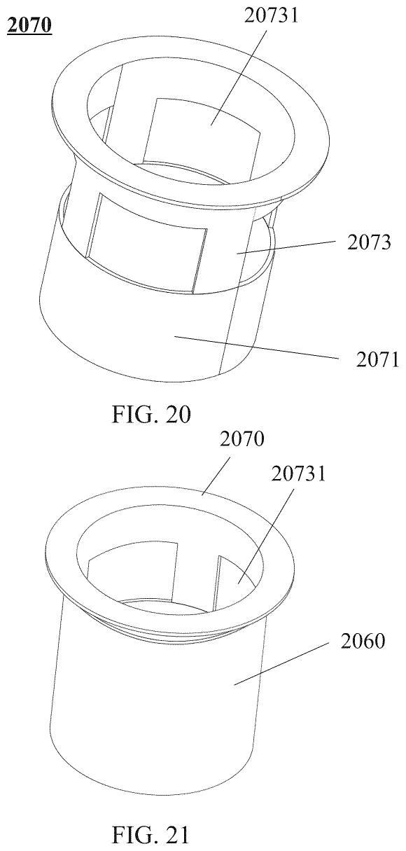

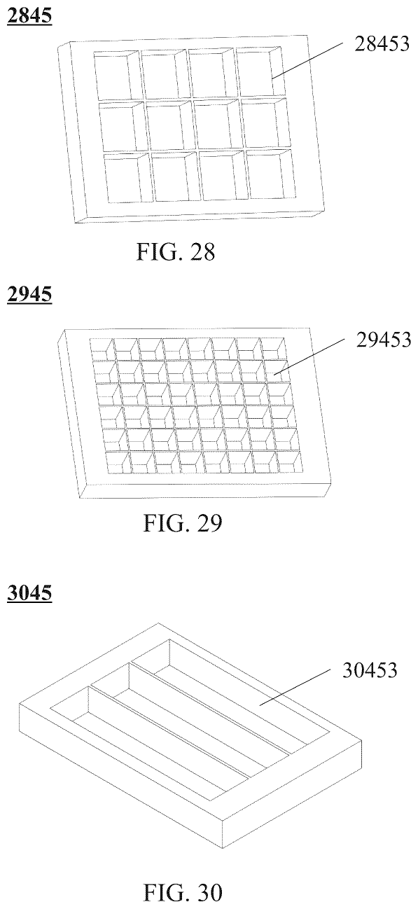

It should be noted that the foregoing description of the culture device 10 is for illustrative purposes only, and does not limit the present disclosure to the scope of the cited embodiments. It is to be understood that for a person skilled in the art, after understanding the principle of the culture device 10, various deformations and modifications can be made to the culture device 10 without departing from this principle. In some embodiments, the culture device 10 may include one or more other components, for example, an upper sealing cover (an upper sealing cover 252 as shown in FIG. 2), a lower sealing cover (a lower sealing cover 251 as shown in FIG. 2), a culture fluid accommodation chamber (a culture fluid accommodation chamber 2845 as shown in FIG. 28), a rack (a rack 2070 as shown in FIG. 20), or the like, or any combination thereof. In some embodiments, one or more of the components of the culture device 10 described above may be omitted. For example, the culture device 10 may not include the sample introduction channel 20, and the sample culture may be directly added to the culture chamber 31. When the count of culture chambers 31 exceeds one, the sample culture may be added well by well or utilizing a batch addition tool (e.g., a row of needles). In some embodiments, a plurality of components of the culture device 10 may be combined into a single component. For example, the culture fluid channel 40 may be integrated into the chamber layer 30. In some embodiments, a component of the culture device 10 may be split into one or more subcomponents.

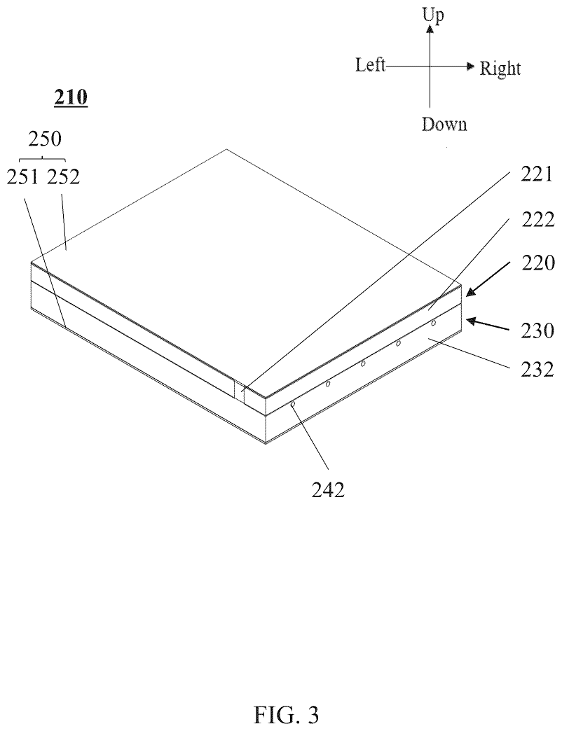

FIG. 2 is a schematic diagram illustrating an exemplary explosion structure of a culture device according to some embodiments of the present disclosure. FIG. 3 is a schematic diagram illustrating an exemplary structure of the culture device illustrated in FIG. 2 after being assembled. A culture device 210 is an exemplary embodiment of the culture device 10 illustrated in FIG. 1.

As shown in FIG. 2 and FIG. 3, the culture device 210 may include a sealing cover 250 (e.g., the upper sealing cover 252 and/or the lower sealing cover 251), a sample introduction channel 220, a chamber layer 230, and a culture fluid channel 240 integrated into the chamber layer 230.

The upper sealing cover 252 may be provided above the sample introduction channel 220. The upper sealing cover 252 may be used to prevent leakage of a culture from the sample introduction channel 220. On the other hand, the upper sealing cover 252 may also prevent other substances (e.g., dust) from entering the sample introduction channel 220 and protecting the sample introduction channel 220 from being damaged by knocks.

The lower sealing cover 251 may be provided below the chamber layer 230. The lower sealing cover 251 may be used to mate with the chamber layer 230 to form at least one culture chamber 231, and may also prevent substances such as dust from entering the culture chamber 231. It should be noted that, as shown in FIG. 2, up and down directions in the present embodiment are parallel to the thickness direction h of the chamber layer, and the thickness direction h of the chamber layer is perpendicular to a top surface and a bottom surface of the chamber layer 230. The up direction refers to a direction pointing from the bottom of the culture chamber to the opening of the culture chamber and the down direction refers to a direction opposite to the up direction, pointing from the opening of the culture chamber to the bottom of the culture chamber. For example, when the culture device 210 is placed on a table, the down direction (or the bottom surface) of the culture device 210 points to the ground. In addition, A being provided above B refers to that A is located above B along the up direction. In other words, a direction pointing from B to A that is located above B is the up direction.

In some embodiments, the sample introduction channel 220 may include a sample introduction plate 222 and a culture inlet and outlet channel 221 opened on the sample introduction plate 222. The sample introduction plate 222 may be configured as a plate-like structure capable of mating with the chamber layer 230. The culture inlet and outlet channel 221 on the sample introduction plate 222 may be in flow communication with at least one culture chamber 231 of the chamber layer 230. When adding a sample culture, the culture may be added to the culture chamber 231 via the culture inlet and outlet channel 221. In some embodiments, biocompatible material may be selected for making the sample introduction plate 222, including natural chitosan, sodium alginate, polyethylene glycol, bioceramics, or the like.

Referring to FIG. 2, in some embodiments, there is only one culture inlet and outlet channel 221, and the shape of the culture inlet and outlet channel 221 is curved. For illustrative purposes, FIG. 4 shows a top view of the culture inlet and outlet channel 221 and the chamber layer 230 (only the culture chambers 231 of the chamber layer 230 is shown). As shown in FIG. 4, the curved culture inlet and outlet channel 221 is sequentially in flow communication with the culture chambers 231-1, 231-2, 231-3 . . . 231-11, 231-12, 231-13 . . . and 231-30. An inlet of the culture inlet and outlet channel 221 is provided near a first culture chamber 231-1 in flow communication with the culture inlet and outlet channel 221. An outlet of the culture inlet and outlet channel 221 is provided near the last culture chamber 231-30 in flow communication with the culture inlet and outlet channel 221. The culture may be added only at the inlet of the culture inlet and outlet channel 221, and the culture may be added to the culture chambers 231 connected thereto in turn via the culture inlet and outlet channel 221. This design can eliminate the step of adding sample cultures to a plurality of culture chambers 231 well by well, henceforth effectively improving the culture efficiency. In some embodiments, the culture inlet and outlet channel 221 may be used to simultaneously add the sample culture to the culture chambers 231.

In some embodiments, the shape of the culture inlet and outlet channel 221 may include other forms in addition to a curved shape shown in FIG. 2 and FIG. 4. For example, the culture inlet and outlet channel 221 may have a grid-like shape that realizes flow communication between adjacent culture chambers 231. Taking the culture chamber 231-1 as an example, the culture inlet and outlet channel 221 may realize flow communication between 231-1 and 231-2, as well as 231-12. As another example, the culture inlet and outlet channel 221 may realize flow communication between culture chambers in a direction parallel to a diagonal of the sample introduction plate 222, e.g., with 231-1 as a starting point, the culture inlet and outlet channel 221 realizes flow communication between connects 231-2, 231-12, and then 231-13, 231-11, 231-3.

In some embodiments, a plurality of culture inlet and outlet channels 221 (not shown in the figures) may be provided. The culture chamber 231 may include a plurality of sets of culture chambers, each set of culture chambers may include at least one culture chamber 231. Each culture inlet and outlet channel 221 may be separately connected to a set of culture chambers. For example, a plurality of culture inlet and outlet channels 221 may be provided, and each inlet and outlet channel 221 is connected to culture chambers 231 located in the same row (i.e., a set of culture chambers 231). Same or different cultures may be added to each set of culture chambers through different culture inlet and outlet channels 221. In some embodiments, each of the culture inlet and outlet channels 221 may include independent inlet and outlet for independently transporting the culture to each of the culture inlet and outlet channels 221.

In some embodiments, the sample introduction channel 220 may be provided in other forms in addition to including the sample introduction plate 222 and the culture inlet and outlet channel 221 as described in one or more embodiments above. For example, the sample introduction channel 220 may be a sample introduction tube (not shown in the figures), wherein an outlet of the sample introduction tube is in flow communication with several culture chambers 231, and an inlet of the sample introduction tube is in flow communication with an external delivery device (e.g., a pumping device). An operator may transport the culture to the culture chamber 231 from the external delivery device via the sample introduction tube. In some embodiments, the sample introduction tube may include a plurality of outlets. For example, an end of the sample introduction tube that is connected to the culture chamber 231 has a plurality of branches, each branch may serve as an outlet for adding the sample culture to a plurality of culture chambers 231 simultaneously. In some embodiments, the sample introduction tube may be a flexible tube or a rigid tube. The shape of the sample introduction tube is not limited and may be straight or curved.

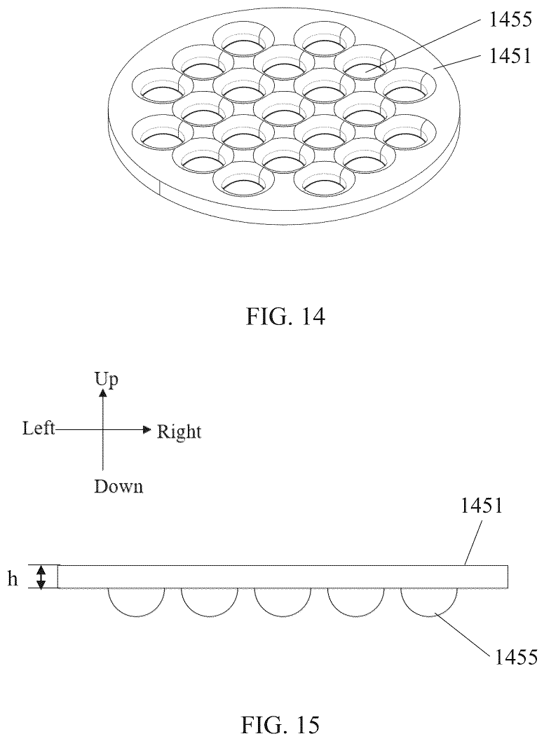

Continuing to refer to FIG. 2, in some embodiments, the chamber layer 230 may include a culture plate 232. The culture plate 232 may be provided with at least one through well 2321. The lower sealing cover 251 may be provided below the culture plate 232 and form the at least one culture chamber 231 with the at least one through well 2321. For example, as shown in FIG. 2, the lower sealing cover 251 may be a flat plate, which may close off a bottom of the through well 2321 to form the culture chamber 231 with a planar bottom surface. In some embodiments, the lower sealing cover 251 may have other forms, which may fit with the through well 2321 to form a culture chamber with a non-planar bottom surface. Merely by way of example, the lower sealing cover 251 may include at least one concave structure, each concave structure may cooperate with a through well 2321 to form a culture chamber with a concave bottom surface. Descriptions of the lower sealing cover can be found elsewhere in the present disclosure (e.g., in the related descriptions of FIGS. 14 and 15), which are not repeated herein.

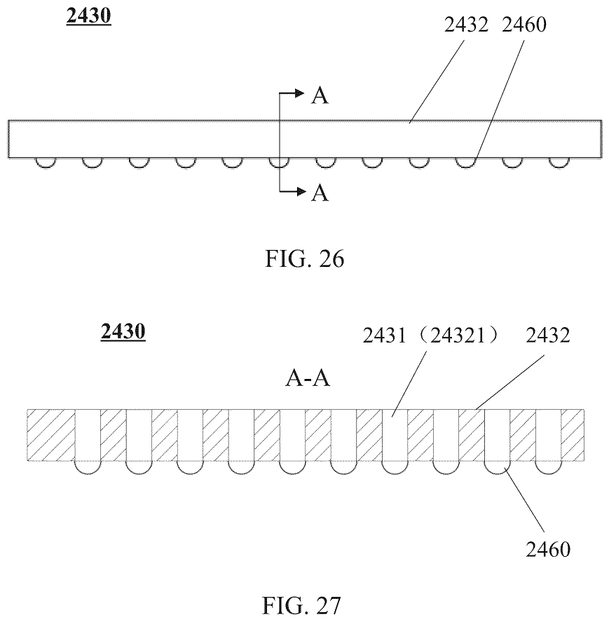

In some embodiments, the culture device 210 may not be provided with the lower sealing cover 251, and the culture plate 232 of the chamber layer 230 may be provided with at least one blind well (not shown in the figures) with an open facing upward (i.e., toward the sample introduction channel 220). Because the bottom of the blind well is closed, the blind well may directly serve as the culture chamber 231 without adding the lower sealing cover 251, which can effectively simplify the structure of the culture device 210. In some embodiments, the bottom of the through well 2321 may be sealed by other structures to form the culture chamber 231. For example, a membrane structure may be provided at the bottom of each through well 2321 to form the culture chamber 231. In some embodiments, the membrane structure may be a porous membrane (e.g., a porous membrane 2460 shown in FIG. 26) to enable a flow of culture fluid and/or other substances (e.g., drugs to be screened) in the culture chamber 231 through the porous membrane. In some embodiments, the porous membrane may be used in conjunction with a culture fluid accommodation chamber shown in FIGS. 28 to 30 (e.g., a culture fluid accommodation chamber 2845 shown in FIG. 28) to update the culture fluid in the culture chamber 231 through the culture fluid accommodation chamber 2845. More details about the porous membrane and the culture fluid accommodation chamber can be found in the description of FIGS. 26-30, which are not repeated here.

Referring to FIG. 2 and FIG. 3, the sample introduction plate 222 may be provided above the culture plate 232, and the sample introduction channel 220 is a separate component from the chamber layer 230. In some embodiments, the culture plate 232 may be assembled and connected to the sample introduction plate 222, e.g., through adhesive bonding, screw connections, or the like. After the sample introduction plate 222 has been assembled with the culture plate 232, the culture may be added to the culture chamber 231 of the culture plate 232 through the culture inlet and outlet channel 221 on the sample introduction plate 222. When a cultivation is finished or no culture needs to be added, the culture plate 232 may be separated from the sample introduction plate 222.

The culture fluid channel 240 is integrated into the chamber layer 30 and configured to add a sample culture fluid to the culture chamber 231 and/or updating the sample culture fluid in the culture chamber 231 (i.e., guiding the sample culture fluid into or out of the culture chamber 231). In some embodiments, the culture chamber 231 may be divided into a plurality of sets, and the culture device 210 may comprise a plurality of culture fluid channels 240. Each culture fluid channel 240 is in flow communication with a set of culture chambers. Each culture fluid channel 240 may include a culture fluid inlet 242, a culture fluid update channel 241, and a culture fluid outlet 243. In the present disclosure, when a plurality of culture fluid channels are provided in the culture device (as shown in FIG. 2), the culture fluid channel may also be referred to as a culture fluid sub-channel; and the culture fluid inlet, the culture fluid update channel, and the culture fluid outlet of the culture channel may also be referred to as a culture fluid sub-inlet, the culture fluid sub-update channel, and a culture fluid sub-outlet, respectively.

In some embodiments, the sample introduction channel 220 may be disposed above the culture fluid channel 240. If the culture fluid channel 240 is provided above the sample introduction channel 220, at least a portion of the culture fluid is located below the culture fluid channel 240, and the at least a portion of the culture fluid fails to be updated through the culture fluid channel 240 in time, therefore, a culture fluid in the culture chamber 231 may not be sufficiently updated or exchanged. To ensure that a sufficient updating of a culture fluid at the bottom of the culture chamber 231, the culture fluid channel 240 may be provided below the sample introduction channel 220. A detailed description of the culture fluid channel 240 can be found elsewhere in the present disclosure (e.g., in the related descriptions of FIGS. 6-13), which is not repeated here.

As shown in FIG. 3, the culture device 210 may be assembled. After being assembled, the sample introduction channel 220 is sealed and only the inlet and outlet of the culture inlet and outlet channel 221 are unsealed (the outlet is not shown in FIG. 3). A bottom of the through well 2321 (not shown in FIG. 3) is sealed by the lower sealing cover 251, hence forming the culture chamber 231. After being assembled, the culture fluid inlet 242 and the culture fluid outlet 243 (not shown in FIG. 3) that are opened on the sides of the chamber layer 30 are unsealed and may be used for adding or updating the sample culture fluid. When the culture device 210 of this embodiment is utilized for cultivating the culture, the culture may be added through the inlet of the culture inlet and outlet channel 221, the culture is sequentially added into several culture chambers 231 through the culture inlet and outlet channel 221, and then excess cultures are discharged through the outlet of the culture inlet and outlet channel 221. In some embodiments, a plurality of culture fluid channels 240 may be utilized to add the sample culture fluid to different sets of culture chambers 231 or update the sample culture fluid, thereby forming control sets. For example, cultures of different concentrations and/or compositions are added to the culture fluid update channel 241 via a designated culture fluid inlet 242, respectively, and then the cultures are added to corresponding culture chambers 231 via the culture fluid update channel 241.

FIG. 5 is a schematic diagram illustrating an exemplary culture device according to some embodiments of the present disclosure. As shown in FIG. 5, a culture device 510 may include the sample introduction channel 220, a chamber layer 530, and a culture fluid channel disposed in the chamber layer 530. FIG. 5 only illustrates the culture fluid inlet 242 of the culture fluid channel. The chamber layer 530 is similar to the chamber layer 230 shown in FIG. 2, and the only difference is that a culture plate 532 is provided with a culture chamber(s) 531 that is formed directly by a blind well rather than a through well.

In addition, different from the culture device 210, only one culture fluid inlet 242 is provided in the culture device 510 for transporting a culture fluid to all culture chambers 531. Specifically, the culture fluid inlet 242 may realize flow communication between a side wall of the culture plate 532 and one of the culture chambers 531 in the form of a through hole, and a culture fluid update channel (not shown in FIG. 5) may connect all the culture chambers 531. An operator may transport the culture fluid to all of the culture chambers 531 through the culture fluid inlet 242.

It is to be noted that the shape, size, and position of the various components of a culture device (e.g., the culture device 210, the culture device 510) shown in FIGS. 2-5 and the descriptions thereof described above are for the purpose of illustration only and do not limit the present description to be limited to the scope of the cited embodiments. It will be appreciated that, for a person skilled in the art, after understanding the principle of the culture device, it may be possible to make various deformations and modifications to the culture device without departing from this principle. Taking the sample introduction plate 222 as an example, its shape is not limited to a rectangular body shown in FIGS. 2-5, but may also be a square plate, a circular plate, a triangular plate, and other regular or irregular shapes. As another example, the sample introduction plate 222 may be provided integrally with the culture plate 232. As a further example, the shapes and dimensions (e.g., lengths and widths) of the sample introduction plate 222 and the culture plate 232 may be the same or different. It will be appreciated that the other components of the culture device 210, like the sample introduction plate 222, may be disposed in other forms as well, which are not repeated herein.

FIG. 6 is a schematic diagram illustrating an exemplary chamber layer and an exemplary culture fluid channel according to some embodiments of the present disclosure. FIG. 7 is a schematic diagram illustrating an exemplary bottom view of the chamber layer and culture fluid channel illustrated in FIG. 6. In practice, the chamber layer may be placed in the form shown in FIG. 6, and the chamber layer shown in FIG. 7 may be present by inverting the chamber layer.

As shown in FIG. 6 and FIG. 7, culture chambers 231 of the chamber layer 230 are divided into five sets with each set including six culture chambers 231. Five culture fluid channels 240-1 (or referred to as culture fluid sub-channels) are integrally provided in the chamber layer 230, and each of the culture fluid channels 240-1 may correspond to a set of culture chambers. Each culture fluid channel 240-1 may include a separate culture fluid inlet 242 (or referred to as a culture fluid sub-inlet), a culture fluid outlet 243 (or referred to as a culture fluid sub-outlet), and a culture fluid update channel 241 (or referred to as a culture fluid sub-update channel).

Merely by way of example, referring to FIG. 6, a set of culture chambers may include a first culture chamber proximate to a sidewall A of the culture plate 232, a second culture chamber proximate to a sidewall B of the culture plate 232, and four third culture chambers disposed between the first culture chamber and the second culture chamber. The culture fluid inlet 242 corresponding to the set of culture chambers may realize flow communication between the first culture chamber and the sidewall A. The culture fluid outlet 243 corresponding to the set of culture chambers may realize flow communication between the second culture chamber and the sidewall B. The culture fluid update channel 241 corresponding to the set of culture chambers may realize flow communication between the first culture chamber and the four third culture chambers in turn, and realize flow communication between the second culture chamber and the third culture chamber.

In some embodiments, the culture fluid outlet 243 and the culture fluid inlet 242 are provided proximate to a top surface of the culture plate 232 shown in FIG. 6. The culture fluid update channel 241 is provided at a location proximate to a bottom surface of the culture plate 232 shown in FIG. 6. In such cases, a horizontal height of the culture fluid outlet 243 may be higher than a horizontal height of the culture fluid update channel 241, which allows for a more adequate addition or update of the culture fluid and/or other substances in the culture chamber. In addition, the culture fluid outlet 243, the culture fluid inlet 242, and the culture fluid update channel 241 may be provided at other locations as long as the horizontal height of the culture fluid outlet 243 at the other locations is higher than the horizontal height of the culture fluid outlet 243. More details regarding the locations of the culture fluid outlet, the culture fluid inlet, and the culture fluid update channel can be found in other embodiments of the present disclosure (e.g., the embodiments of FIGS. 8-13).

In some embodiments, the culture fluid outlet 243, the culture fluid inlet 242, and the culture fluid update channel 241 may be in the form of a pipe. For example, in the embodiment shown in FIG. 6 and FIG. 7, the culture fluid outlet 243 realizes flow communication between the second culture chamber and the sidewall B in the form of a pipe. The culture fluid inlet 242 realizes flow communication between the first culture chamber and the sidewall A in the form of a pipe. The culture fluid update channel 241 realizes flow communication between the first culture chamber, the four third culture chambers, and the second culture chamber sequentially in the form of a pipe.

In some embodiments, the culture fluid channel 240-1 may have a screening function that allows only specific substances to pass through. For example, the culture fluid channel 240-1 may allow only the culture fluid and/or a drug (e.g., a drug to be screened) to be guided in or out of the culture chamber 231 without allowing a culture to pass through (i.e., the channel may retain the culture). In some embodiments, dimensional parameters of the culture fluid channel 240-1 may be adjusted or a specific component may be provided in the culture fluid channel 240-1 to enable the culture fluid channel 240-1 to have a screening function.

For example, the culture fluid outlet 243 may have a first connection 245 with the at least one culture chamber 231. The culture fluid update channel 241 may have a second connection 247 with the at least one culture chamber 231. The first connection 245 may include a contact surface formed when the culture fluid outlet 243 is in connection with the culture chamber 231, and the second connection 247 may include a contact surface formed when the culture fluid update channel 241 is in connection with the culture chamber 231. In some embodiments, cross-sectional sizes of the first connection 245 and/or the second connection 247 are smaller than a size of a component of the culture, hence preventing the culture in the culture chamber 231 from flowing out of the culture chamber 231 via the culture fluid channel 240. For example, the cross-sectional sizes of the first connection 245 and/or the second connection 247 may be smaller than a size of a single cell (e.g., 5 micrometers) when the culture is a cell. For example, when the culture is a cell mass, the cross-sectional sizes of the first connection 245 and/or the second connection 247 may be smaller than a size of a single cell mass. Merely by way of example, the size of the single cell mass is in a range of 50 micrometers to 4 millimeters, and the cross-sectional size may be determined based on the size of the cell mass.

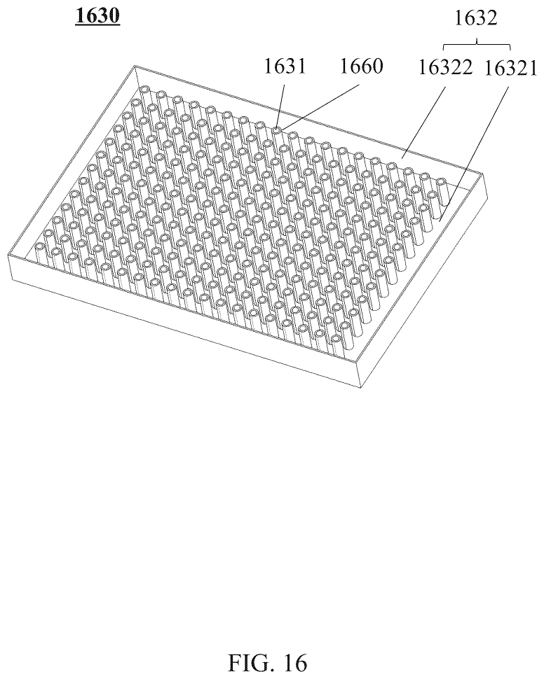

In some embodiments, the first connection 245 and/or the second connection 247 may be provided with a porous membrane for retaining the culture. The porous membrane is a membrane that is disposed with one or more pore structures. The porous membrane may utilize its pore structure to allow specific substances to pass through while other substances may be retained. For example, when a size of an object is larger than a pore diameter of the porous membrane, the object may be trapped by the porous membrane and may not be able to pass through the porous membrane. When a size of an object is smaller than the pore diameter of the porous membrane, the object may be able to pass through the porous membrane. The pore diameter of the porous membrane is a size of the pore structure that is disposed on the porous membrane. In some embodiments, the pore diameter of the porous membrane disposed at the first connection 245 and/or the second connection 247 is related to a size of the culture. For example, the pore size of the porous membrane may be smaller than the size of a single cell (e.g., 5 micrometers) when the culture is a cell. When the culture is a cell mass, the pore size of the porous membrane may be smaller than the size of a single cell mass. By providing the porous membrane at the first connection 245 and/or the second connection 247, the culture may not flow out when the culture fluid or other substances are added or updated. More details regarding the porous membrane can be found in the description of FIGS. 16-18, which are not repeated here.

In some embodiments, when the culture is utilized for drug screening, the culture fluid may be guided out from the culture chamber 231 and then the drug to be screened may be added. For example, a pipette gun is connected to the culture outlet 243 to aspirate the culture fluid, or the pipette gun is connected to the culture fluid inlet 242 to add the drug to be screened. In a process of draining the culture fluid from the culture chamber 231 and adding the drug to be screened into the culture chamber 231, since a dimension of a constituent unit of the culture fluid and the drug to be screened is smaller than the pore size of the porous membrane and a size of the culture is larger than the pore size of the porous membrane, the culture in the culture chamber 231 may be retained by the porous membrane (i.e., the culture is retained in the culture chamber 231), and the culture fluid and the drug to be screened may pass through the porous membrane. By providing the porous membrane, the culture fluid and the drug to be screened can be drained without changing the culture chamber (i.e., transferring the culture to other culture chambers), and the loss of the culture can be effectively avoided. Further, a device such as the pipette gun can be operated only by being connected to the culture fluid inlet 242 and the culture fluid outlet 243, which also prevents the device such as the pipette gun from directly connecting to the culture chamber 231, henceforth improving the safety and reliability of the process.

FIG. 8 is a schematic diagram illustrating an exemplary chamber layer and an exemplary culture fluid channel disposed on a top surface of the chamber layer according to some embodiments of the present disclosure. A culture fluid channel 240-2 shown in FIG. 8 is similar to the culture fluid channel 240-1 of FIG. 6, the difference is that the culture fluid inlet 242, the culture fluid outlet 243, and the culture fluid update channel 241 are all provided on a top surface of the chamber layer 230. For example, the culture fluid inlet 242, the culture fluid outlet 243, and the culture fluid update channel 241 may be grooves disposed on a top surface of the culture plate 232.

FIG. 9 is a schematic diagram illustrating an exemplary chamber layer and an exemplary culture fluid channel disposed on a bottom surface of the chamber layer according to some embodiments of the present disclosure. A culture fluid channel 240-3 shown in FIG. 9 is similar to the culture fluid channel 240-2 shown in FIG. 8, and the difference is that the culture fluid inlet 242, the culture fluid outlet 243, and the culture fluid update channel 241 are all provided on a bottom surface of the culture plate 232. For example, the culture fluid inlet 242, the culture fluid outlet 243, and the culture fluid update channel 241 may be grooves provided on the bottom surface of the culture plate 232.

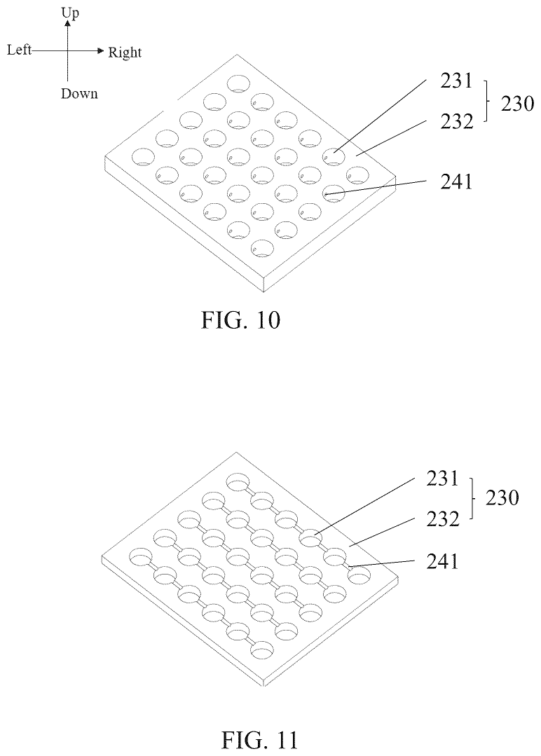

FIG. 10 is a schematic diagram illustrating an exemplary chamber layer and an exemplary culture fluid channel disposed inside the chamber layer illustrated in FIG. 10 according to some embodiments of the present disclosure. FIG. 11 is a schematic diagram illustrating an exemplary cross-sectional structure of the chamber layer illustrated in FIG. 10. As shown in FIG. 10 and FIG. 11, the culture fluid update channel 241 may be disposed on a certain horizontal cross-section within the culture plate 232. The horizontal cross-section refers to a plane parallel to a top surface and/or a bottom surface of the culture plate 232.

In some embodiments, a culture fluid inlet (not shown in FIG. 10 and FIG. 11) and a culture fluid outlet (not shown in FIG. 10 and FIG. 11) may be provided at any location in the chamber layer 230 as long as a horizontal height of the culture fluid outlet is not lower than a horizontal height of the culture fluid update channel 241. That is, the culture fluid outlet is located at the same horizontal cross-section as the culture fluid update channel 241, or the culture fluid outlet is located above the culture fluid update channel 241, so as to ensure that a culture fluid in the culture chamber 231 has a certain fluid level height. The horizontal height here may be represented by a distance from an object to a bottom surface of the culture chamber 231 or to a bottom surface of the culture plate 232. For example, a distance from the culture fluid outlet to the bottom surface of the culture chamber 231 is one-third of a depth of the culture chamber 231, and a distance from the culture fluid update channel 241 to the bottom surface of the culture chamber 231 is one-fourth of the depth of the culture chamber 23, then the culture fluid outlet is located above the culture fluid update channel 241.

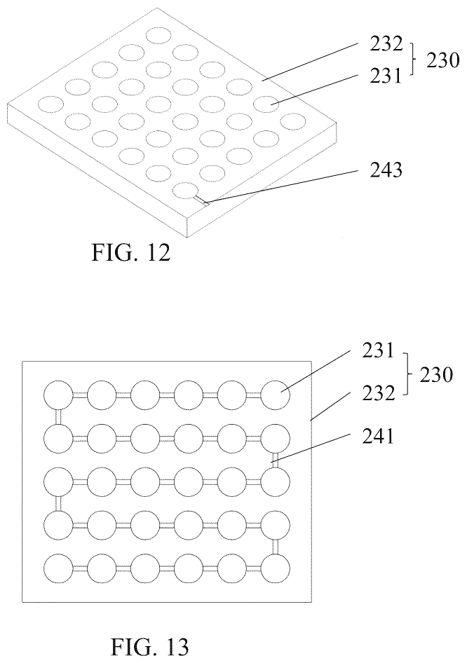

FIG. 12 is a schematic diagram illustrating an exemplary chamber layer according to some other embodiments of the present disclosure. FIG. 13 is a schematic diagram illustrating an exemplary cross-sectional structure of the chamber layer illustrated in FIG. 12. As shown in FIG. 12 and FIG. 13, the culture fluid outlet 243 may be provided on a top surface of the chamber layer 230. The culture fluid update channel 241 and a culture fluid inlet (not shown in the figures) may be provided inside the chamber layer 230. For example, a distance from the culture fluid inlet to a bottom surface of the culture chamber 231 is one-third of a depth of the culture chamber 231, and a distance from the culture fluid update channel 231 to the bottom surface of the culture chamber 231 is one-fifth of the depth of the culture chamber 231.

FIG. 14 is a schematic diagram illustrating an exemplary lower sealing cover according to some embodiments of the present disclosure. FIG. 15 is a schematic diagram illustrating an exemplary side view of the lower sealing cover illustrated in FIG. 14. A lower sealing cover 1451 shown in FIG. 14 and FIG. 15 has a shape of a circular plate. It will be appreciated that the circular plate shape is for illustrative purposes only and is not intended to limit the shape of the lower sealing cover 1451. The lower sealing cover 1451 may have other shapes, e.g., the lower sealing cover 1451 may be a rectangular shape for cooperating with the culture plate 232 of FIG. 2.

As shown in FIG. 14 and FIG. 15, the lower sealing cover 1451 may have at least one concave structure 1455. When the lower sealing cover 1451 is provided on the bottom surface of a culture plate comprising at least one through well, the at least one through well may cooperate with the at least one concave structure 1455 to form at least one culture chamber. Merely by way of example, when the lower sealing cover 1451 is provided on the bottom surface of the culture plate 232, the at least one through well 2321 is capable of cooperating with the at least one concave structure 1455 to form the at least one culture chamber. The bottom of the culture chamber formed by the lower sealing cover 1451 and the culture plate 232 is a downwardly concave surface.

In some embodiments, the concave structure 1455 may include a porous membrane. The porous membrane makes the concave structure capable of retaining substances whose sizes are larger than a pore size of the porous membrane. For example, a porous membrane with a specific pore size may be used such that a culture fluid or other substance may pass through the concave structure 1455, and a culture in the culture chamber may not pass through the concave structure 1455. More details regarding the porous membrane can be found in the description of FIGS. 16-22, which are not repeated here.

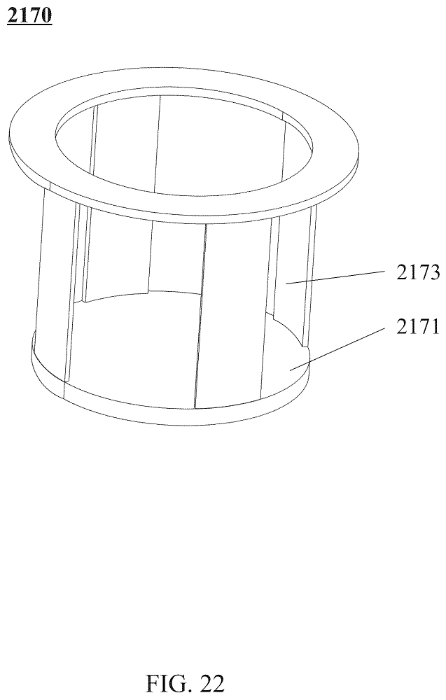

In some embodiments, an inner wall of the concave structure 1455 may have cell adhesion inertia such that cultures within the concave structure 1455 may aggregate into clusters more quickly, thereby improving culture efficiency. In some embodiments, the concave structure 1455 may be rendered cell adhesion inertia by providing a membrane of inert material on the inner wall of the concave structure 1455. More details regarding the membrane of inert material can be found in the description of FIG. 22, which are not repeated here.

The present disclosure also provides a culture device that includes a culture chamber. When the culture device is utilized for updating the culture fluid or for drug screening, other substances or the culture fluid in the culture chamber may be guided in or out of the culture chamber, and the culture is retained in the culture chamber, which effectively avoids affecting the culture when a sample culture is added to the culture chamber or the sample culture in the culture chamber is updated.

FIG. 16 is a schematic diagram illustrating an exemplary chamber layer according to some embodiments of the present disclosure. A chamber layer 1630 shown in FIG. 16 is an exemplary embodiment of the chamber layer 30 shown in FIG. 1. The chamber layer 1630 may include a culture plate 1632 and at least one porous membrane 1660 disposed on the culture plate 1632. The culture plate 1632 includes a bottom plate 16321 and a side plate 16322. The side plate 16322 is provided around the bottom plate 16321 and forms an accommodation space with the bottom plate 16321. The accommodation space may be used to accommodate the porous membrane 1660. The porous membrane 1660 may be used to form the culture chamber 1631 with the culture plate 1632. The porous membrane 1660 may be attached to or may be used to form a sidewall of the at least one culture chamber 1631.

In some embodiments, the porous membrane 1660 may allow only specific substances to pass through for retaining other substances. For example, when a dimension of a constitution unit of a culture fluid or other substances (e.g., a drug) is smaller than a pore size of the porous membrane 1660, and a dimension of a constitution unit of a culture is larger than the pore size of the porous membrane 1660, the culture fluid or other substances may pass through the porous membrane 1660, while the culture may fail to pass through the porous membrane 1660 and be retained in the culture chamber. In this case, when a culture fluid channel (not shown in FIG. 16) is utilized to update the culture fluid or other substances, the culture may not be lost through the porous membrane 1660, and a cultivation condition in the culture chamber 1631 may be undisturbed, which can improve the stability of a cultivation system.

The pore size of the porous membrane 1660 may be set according to a cultivation need. For example, the pore size of the porous membrane 1660 may be set with reference to the porous membranes described in FIG. 6 and FIG. 7. As another example, the pore size of the porous membrane 1660 may be different from a pore size of the porous membrane described in FIG. 6 and FIG. 7. In some embodiments, the pore size of the porous membrane 1660 may range from 0.1 nanometers to 1 centimeter. In some embodiments, when the porous membrane 1660 is used to retain small molecules, such as sodium ions, glucose, lactate, ammonia ions, or the like, a range of pore size of the porous membrane 1660 may be in a range of 0.1 nanometers to 1 nanometer. In some embodiments, when the porous membrane 1660 is used to retain macromolecules, such as proteins, polysaccharides, DNA, etc., a range of pore size of the porous membrane 1660 may be in a range of 1 nanometer to 100 nanometers. In some embodiments, when the porous membrane 1660 is used to retain particulate matter, latex, micelles, etc., the pore size of the porous membrane 1660 may be in a range of 5 nanometers to 1 micron. In some embodiments, when the porous membrane 1660 is used to retain biological tissues such as viruses, bacteria, mycoplasmas, cells, and cellular exosomes, etc., a range of pore size of the porous membrane 1660 may be in a range of 100 nanometers to 10 micrometers. In some embodiments, when the porous membrane 1660 is used to retain biological tissue aggregates such as organoids, cell clusters, embryos, isolated tissues and organs, or the like, the pore size of the porous membrane 1660 may be in a range of 10 microns to 1 cm.

In some embodiments, the porous membrane 1660 may include at least one of a hollow fiber membrane, a tubular membrane, a ceramic membrane, or a polymer membrane.

In some embodiments, the shape of the culture chamber 1631 may include other regular or irregular shapes such as a cylindrical chamber, a prismatic chamber, etc., and a specific shape of the culture chamber 1631 is not limited in the present disclosure. In some embodiments, a size of the culture chamber 1631 formed by the porous membrane 1660 and the culture plate 1632 may be set based on the type of the culture. For example, the type of culture may include a single cell, a cluster of single cells, a single organoid, isolated tissues and organs, or the like. Since different types of cultures have different sizes, the sizes of the culture chamber 1631 may be different. In some embodiments, the size of the culture chamber 1631 may be represented by a size of an inscribed circle of the culture chamber 1631. In some embodiments, an inner diameter (i.e., a diameter) of the inscribed circle of the culture chamber 1631 may be in a range of 5 micrometers to 10 micrometers when the culture is a single cell. In some embodiments, when the culture is a cluster of single cells, the inner diameter of the inscribed circle of the culture chamber 1631 may be in a range of 10 micrometers to 1000 micrometers. In some embodiments, when the culture is a single organoid (e.g., a brain organoid), the inner diameter of the inscribed circle of the culture chamber 1631 may be in a range of 100 micrometers to 5 centimeters. In some embodiments, when the culture is isolated tissues and organs, etc., the inner diameter of the inscribed circle of the culture chamber 1631 is in a range of 1 centimeter to 1 meter. The culture chamber 1631 in this embodiment may be used to accommodate cultures of any size. The larger the size of a culture to be accommodated, the larger the inner diameter of the inscribed circle of the culture chamber 1631, and thus there is no limitation on a maximum value of the inner diameter of the inscribed circle of the culture chamber 1631.

In some embodiments, the culture chamber 1631 may be a cylindrical chamber with a ratio of a length (i.e., depth) to a diameter in a certain range. The range may be 0.5 to 60, 0.75 to 40, 1 to 20, 2 to 10, or the like.

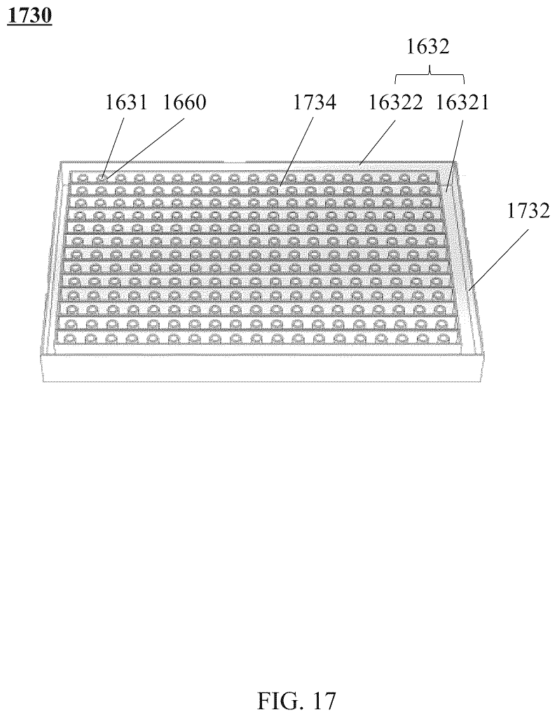

FIG. 17 is a schematic diagram illustrating an exemplary chamber layer according to some embodiments of the present disclosure. A chamber layer 1730 shown in FIG. 17 is similar to the chamber layer 1630 shown in FIG. 16, and the difference is that the chamber layer 1730 further includes a first chamber baffle 1732 and a second chamber baffle 1734. The first chamber baffle 1732 may enclose all culture chambers 1631. The second chamber baffle 1734 may separate two adjacent rows of culture chambers 1631, thereby separating the culture chambers 1631 by sets. Referring to FIG. 17, the culture chambers 1631 are surrounded by four first chamber baffles 1732 to form a chamber layer space, in which all culture chambers 1631 are accommodated. The chamber layer space is further divided into 13 sub-chamber layer spaces by 12 second chamber baffles 1734, and the culture chambers 1631 are divided into 13 sets, with each sub-chamber layer space containing a set of culture chambers and each set of culture chambers containing 19 culture chambers 1631.

In some embodiments, a plurality of culture chambers may be used to form a plurality of control sets. For example, different cultures may be added to different sets of culture chambers during a cell cultivation. As another example, when drug screening is performed after the cell cultivation is completed, drugs of different compositions or drugs of the same compositions and different concentrations may be added into the various sets of culture chambers for high-through drug screening. In some embodiments, the chamber layer 1630 may be combined with a culture channel described elsewhere in the present disclosure, such as the culture channel 240-1 shown in FIG. 6, to update the culture fluid by sets. Specifically, a plurality of culture fluid sub-channels of the culture fluid channel may be separately in flow communication with each set of culture chambers of the chamber layer 1630. For example, one culture fluid sub-channel of the culture fluid channels may be in flow communication with one set of culture chambers of the chamber layer 1630, i.e., a sub-chamber layer space serves as a sub-update channel, and through a culture fluid sub-inlet and a culture fluid sub-outlet independently provided on the sub-chamber layer space, the culture fluid and/or drug in the set of culture chambers accommodated in the sub-chamber layer space may be updated. In addition, the culture fluid/drug in other sets of culture chambers may also be updated through corresponding culture fluid sub-channels, and the update of the culture fluid and/or drug between each set does not affect the other.

In some embodiments, the culture chambers 1631 may be divided into any count of control sets. Each set of culture chambers may contain any count of culture chambers 1631. A count of culture chambers 1631 in the different sets of culture chambers may be the same or different. For example, the count of culture chambers 1631 in each set of culture chambers may be the same and in a certain range (e.g., in a range of 1 to 50, in a range of 5 to 30, in a range of 10 to 20, etc.). As another example, the culture chambers 1631 may include 2 sets of culture chambers, one set of which may include 20 culture chambers 1631 and the other set of which may include 30 culture chambers 1631.

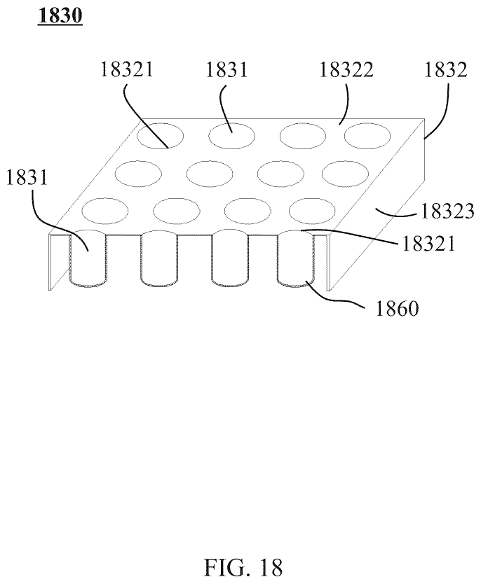

FIG. 18 is a schematic diagram illustrating an exemplary chamber layer according to some embodiments of the present disclosure. A chamber layer 1830 shown in FIG. 18 is an exemplary embodiment of the chamber layer 30 shown in FIG. 1. As shown in FIG. 18, the chamber layer 30 may include a culture plate 1832 and a porous membrane 1860. The culture plate 1832 includes a horizontal plate 18322 and a side enclosure plate 18323 provided around the horizontal plate 18322. A plurality of through wells 18321 may be provided on the horizontal plate 18322 of the culture plate 1832. The porous membrane 1860 may be provided at a bottom of the through well 18321, thereby forming an entire culture chamber 1831, i.e., sidewalls and a bottom wall of the culture chamber 1831 are formed by the porous membrane 1860.

Referring to FIG. 18, the porous membrane 1860 may be a cartridge structure with a concave bottom and an opening(s) at the top, i.e., a bottom of the cartridge structure of the porous membrane 1860 protrudes away from an internal chamber of the porous membrane 1860. A sidewall of the porous membrane 1860 may be connected with a bottom surface of the horizontal plate 18322, such that the porous membrane 1860 is in flow communication with the through well 18321 to form the culture chamber 1831 with an opening at the top. In some application scenarios, a culture fluid or other substances (e.g., a drug to be screened) may be added to the culture chamber 1831 from an underside of the culture plate 1832, and the culture fluid or other substances may pass through the porous membrane 1860 into the culture chamber 1831. At the same time, the culture fluid or other substances may be discharged from the culture chamber 1831 via the porous membrane 1860 to enable the update and exchange of substances.

In some embodiments, a culture fluid accommodation chamber such as that shown in FIGS. 28-30 may be provided below the culture plate 1832, e.g., a culture fluid accommodation chamber 2845 shown in FIG. 28, a culture fluid accommodation chamber 2945 shown in FIG. 29, and a culture fluid accommodation chamber 3045 shown in FIG. 30. For example, at least a portion of the culture fluid chamber 1831 may be placed within the culture fluid accommodation chamber 2945 shown in FIG. 29. The culture fluid or other substances may be added to the culture fluid accommodation chamber 2945, and the culture fluid or other substances accommodated in the culture fluid accommodation chamber 2945 may enter the culture chamber 1831 via the porous membrane 1860. In some embodiments, a bottom of the porous membrane 1860 may also be planar. The culture chamber with a concave bottom formed by the porous membrane 1860 is more suitable for three-dimensional cultivation, while a culture chamber with a planar bottom formed by the porous membranes 1860 is more suitable for two-dimensional cultivation.

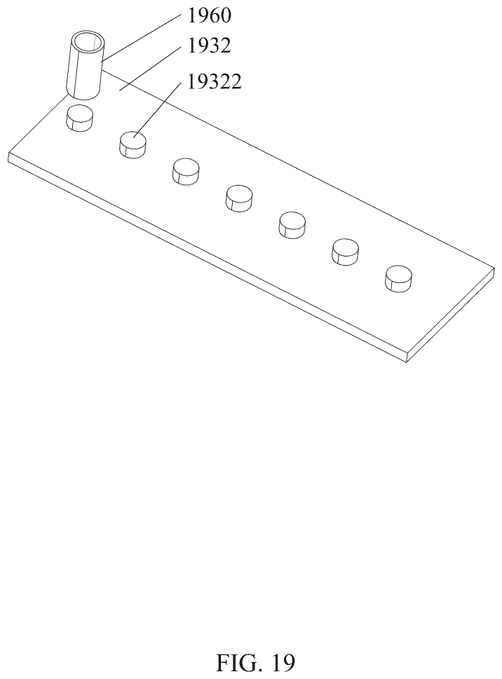

FIG. 19 is a schematic diagram illustrating an exemplary porous membrane assembled with a culture plate according to some embodiments of the present disclosure. As shown in FIG. 19, a porous membrane 1960 may be designed as a tubular structure. A positioning block 19322 is disposed on a surface of a culture plate 1932, and a shape of the positioning block 19322 is adaptable to an internal chamber of the porous membrane 1960 of the tubular structure. During the assembly, the positioning block 19322 may be embedded in the internal chamber of the porous membrane 1960 to form a tubular culture chamber (e.g., the culture chamber 1631 in FIG. 16).

In some embodiments, the porous membrane 1960 may be embedded into the positioning block 19322. For example, the positioning block 19322 may be provided with an annular positioning groove (not shown in the figures). The positioning groove may be adapted with the porous membrane 1960 such that the porous membrane 1960 may be embedded in the positioning groove. The positioning block 19322 may be used as a bottom wall of a culture chamber to form the culture chamber with the porous membrane 1960 (not shown in the figures), and the positioning block 19322 may also prevent the porous membrane 1960 from displacement and improve the connection stability between the porous membrane 1960 and the culture plate 1932.

In some embodiments, the positioning block 19322 may be a portion of the culture plate 1932, i.e., the positioning block 19322 may be integrally molded with the culture plate 1932. In some embodiments, the positioning block 19322 and the culture plate 1932 may be separately molded and then assembled. For example, the positioning block 19322 and the culture plate 1932 may be connected by snap-fit, bonding, etc. In some embodiments, the positioning block 19322 may be made of a biocompatible material. More details regarding the biocompatible material can be found in the description of FIG. 2, which are not repeated here. In some embodiments, the connection stability between the culture plate 1932 and the porous membrane 1960 can be improved in other ways in addition to the positioning block 19322. For example, at least one blind well (not shown in the figures) adapted to fit the porous membrane 1960 may be provided on the culture plate 1932, and the porous membrane 1960 may be embedded in the blind well to form the culture chamber.

In some embodiments, the positioning block 19322 may be omitted, and the culture chamber may be formed after the porous membrane 1960 is attached to the culture plate 1932. For example, the porous membrane 1960 is a hollow tubular structure and the culture plate 1960 is a flat plate structure. A culture chamber with a closed bottom end and an open upper end may be formed by placing the porous membrane 1960 on a surface of the culture plate 1932, the porous membrane 1960 may be used as a sidewall of the culture chamber, and the culture plate 1932 may serve as a bottom wall of the culture chamber. In some embodiments, the porous membrane 1960 may form the sidewall and the bottom wall of the culture chamber. For example, the porous membrane 1960 is a cartridge structure (similar to that shown in FIG. 18) with a closed end and an open end, wherein the closed end of the membrane is connected to the culture plate 1932, and an internal chamber of the cartridge structure may serve as the culture chamber. In some embodiments, the porous membrane 1960 and the culture plate 1932 may be physically connected, for example, by bonding the porous membrane 1960 to the culture plate 1932.

FIG. 20 is a schematic diagram illustrating an exemplary rack according to some embodiments of the present disclosure. FIG. 21 is a schematic diagram illustrating an exemplary rack illustrated in FIG. 20 after being assembled with a porous membrane. As shown in FIG. 20 and FIG. 21, a culture device, such as the culture device 10 shown in FIG. 1, may include a rack 2070 to which a porous membrane 2060 is attached. The rack 2070 may include a first support portion 2071 and a second support portion 2073 attached to each other. The second support portion 2073 may be an internally hollow circular structure, and a peripheral wall of the second support portion 2073 may be provided with a skeletonized structure 20731. The first support portion 2071 may be an internally hollow circular structure (or an internally non-hollow cylindrical structure) that does not have a skeletonized structure on the peripheral wall thereof.

Referring to FIG. 21, the porous membrane 2060 may be attached to an outer peripheral wall of the first support portion 2071 and the second support portion 2073, and is supported by the first support portion 2071 and the second support portion 2073. In some embodiments, the porous membrane 2060 may also be attached to an inner peripheral wall of the second support portion 2073. Optionally, when the first support portion 2071 is a circular structure, the porous membrane 2060 may also be attached to an inner peripheral wall of the first support portion 2071. Compared with directly placing the porous membrane 2060 on a culture plate (such as the culture plate 1932 shown in FIG. 19), attaching the porous membrane 2060 to the rack 2070 and then connecting the rack 2070 to the culture plate may avoid deformation of the porous membrane 2060 and effectively prevent the porous membrane 2060 from displacement relative to the culture plate. In some embodiments, the rack 2070 and the culture plate may be connected by bonding, winding, or the like. For example, the porous membrane 2060 may be bonded to a portion of the second support portion 2073 other than the skeletonized structure 20731.

The skeletonized structure 20731 may provide a channel for the flow of a culture fluid or other substances (e.g., a drug to be screened). Specifically, after the porous membrane 2060 is attached to the rack 2070, the culture fluid or other substances in the culture chamber (e.g., the culture chamber 31 as illustrated in FIG. 1) may be discharged from the skeletonized structure 20731 on the porous membrane 2060. The culture fluid or other substances may also be transported into the culture chamber through the porous membrane 2060 and the skeletonized structure 20731.

In some embodiments, when the first support portion 2071 is the internally non-hollow cylindrical structure, the porous membrane 2060 may be attached to the peripheral wall of the second support portion 2073 to form a sidewall of the culture chamber, and the first support portion 2071 may be used directly as a bottom wall of the culture chamber. When the first support portion 2071 is the internally hollow circular structure, the porous membrane 2060 may be attached to a bottom of the first support portion 2071 to form a bottom of the culture chamber. In some embodiments, a surface in the first support portion 2071 used to form the bottom of the culture chamber may be concave. For example, the surface may be a downwardly concave surface, i.e., the surface protrudes downward away from an open end of the rack 2070. As another example, the first support portion 2071 may be an upwardly concave surface, i.e., the surface protrudes upward toward the open end of the rack 2070. In some embodiments, a culture chamber with a concave bottom surface is more suitable for a three-dimensional culture model.

In some embodiments, the rack 2070 and the culture plate (e.g., the culture plate 1632 as shown in FIG. 16, and the culture plate 1932 as shown in FIG. 19) may be integrally molded. In some embodiments, the rack 2070 may be mounted to the culture plate. For example, the rack 2070 may be disposed on a top surface of a bottom plate 16321 of the culture plate 1632, and the porous membrane 1660 may be socketed to an outer peripheral wall of the rack 2070. As another example, the rack 2070 may be connected with a bottom surface of the culture plate 1832, wherein one end of the second support portion 2073 away from the first support portion 2071 is connected with a bottom surface of a horizontal plate 18322 and realizes flow communication between an internal chamber of the rack 2070 and the through well 18321 on the culture plate 1832, and the porous membrane 2060 may be attached to an outer peripheral wall of the rack 2070 to form a chamber layer 1830 similar to that shown in FIG. 18. As another example, the first support portion 2071 of the rack 2070 may be connected with the top surface of the culture plate 1932, and the porous membrane 2060 may be attached to the rack 2070 to form a culture chamber (the culture chamber 1631 shown in FIG. 16). In this embodiment, the positioning block 19322 may have an annular positioning groove, and a first support portion 2071 of the porous membrane support 2070 to which the porous membrane 2060 is attached may be embedded in the positioning groove.

FIG. 22 is a schematic diagram illustrating another exemplary rack according to some embodiments of the present disclosure. As shown in FIG. 22, a rack 2170 may include a first support portion 2171 and a second support portion 2173 that is connected to the first support portion 2171. The second support portion 2173 may be the same as or similar to the second support portion 2073 shown in FIG. 20. The first support portion 2171 functions similarly to the first support portion 2071 shown in FIG. 20. A difference is that the first support portion 2171 is a circular plate-like structure with a relatively small thickness. A bottom of a culture chamber formed by a porous membrane (not shown in the figure) attached to the rack 2170 may be planar. A culture chamber with a planar bottom is more suitable for a two-dimensional culture model.

It is to be noted that shapes, sizes, positions, accounts, or the like of a chamber layer (e.g., the chamber layers 1630, 1730, 1830) and related components shown in FIGS. 16-22, and the related descriptions thereof described above are for illustrative purposes only and do not limit the present disclosure to the scope of the cited embodiments. It can be understood that for those skilled in the art, after understanding the principle of the chamber layer and its related parts, various deformations and modifications may be made without departing from this principle.

In some embodiments, the chamber layer may further comprise a membrane of inert material disposed on an inner bottom wall of the culture chamber. For example, when a three-dimensional culture is performed utilizing the culture chamber 1831, there is a need to prevent a culture from adhering to the bottom wall of the culture chamber 1831 (if the culture adheres to the bottom wall of the culture chamber, the culture may form an adherent culture and thus converting the three-dimensional culture into a two-dimensional culture). The membrane of inert material disposed on the inner bottom wall of the culture chamber 1831 effectively prevents adhesion of the culture to the bottom wall. In some embodiments, the membrane of inert material may be used in combination with a porous membrane. For example, in the embodiment shown in FIG. 18, the membrane of inert material may be provided on an inner bottom wall of the porous membrane 1860. As another example, the porous membrane 1860 may be provided with the membrane of inert material on both the bottom and the sidewall. In some embodiments, the membrane of inert material may be made from a material such as polyethylene, polyvinyl chloride, acrylic resin, polytetrafluoroethylene, silicone polymer, or the like. In some embodiments, the material of the porous membrane 1860 may include an inert material, i.e., a composition of the porous membrane 1860 includes an inert material such that the porous membrane 1860 is inherently cell-adhesion inert.

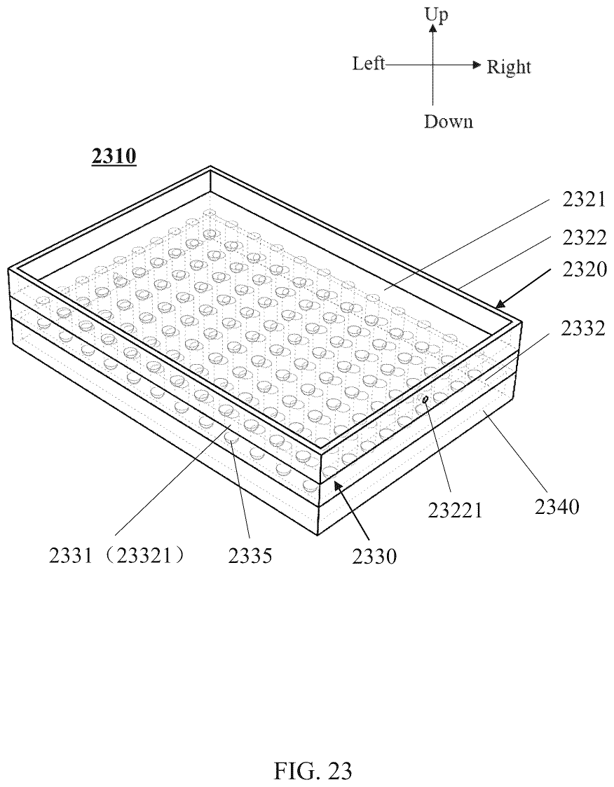

FIG. 23 is a schematic diagram illustrating an exemplary assembled culture device according to some embodiments of the present disclosure. As shown in FIG. 23, a culture device 2310 may include a sample introduction channel 2320, a chamber layer 2330, and a culture fluid channel 2340 that are independent of each other. The sample introduction channel 2320 may be disposed above the chamber layer 2330, and the culture fluid channel 2340 may be disposed below the chamber layer 2330. In this embodiment, a sample culture and a sample culture fluid may be added to the chamber layer 2330 (e.g., the culture chamber(s) 2331 of the chamber layer 2330) through the sample introduction channel 2320 and the culture fluid channel 2340 that are provided independently of each other.

In some embodiments, the sample introduction channel 2320 may include a sample introduction plate 2322 and a culture inlet and outlet channel 2321 provided on the sample introduction plate 2322. Different from the sample introduction channel 221 of FIG. 2, the culture inlet and outlet channel 2321 is a chamber disposed on the sample introduction plate 2322, and a culture may be added to the chamber. For example, the culture may be added to the chamber from a sample introduction port 23221 (an inlet of the culture inlet and outlet channel 2321) of the sample introduction plate 2322. As another example, the culture may be automatically transported into the culture inlet and outlet channel 2321 by an external transporter. The culture then passes through the culture inlet and outlet channel 2321 and enters a culture chamber connected thereto. For example, a plurality of sampling introduction wells (not shown in the figures) may be provided on a bottom of the sample introduction plate 2322, and the sampling introduction wells may be in flow communication with the culture chamber 2331, and a culture entering the chamber may enter the culture chamber(s) 2331 via the sampling introduction wells. In this way, the sample culture may be added to a plurality of culture chambers by introducing the sample culture only once, which can improve the culture efficiency.

The chamber layer 2330 may include a culture plate 2332, a through well(s) 23321 disposed on the culture plate 2332, and a concave structure(s) 2335 disposed below the through well 23321. The concave structure 2335 may close off an underside of the through well 23321 to form the culture chamber 2331 with a concave bottom. As shown in FIG. 23, a bottom of the culture chamber 2331 is a downwardly concave surface protruding away from the through well 23321. In some embodiments, the bottom of the culture chamber 2331 may be an upwardly concave surface protruding toward the through well 23321. By providing the concave structure 2335, aggregation of cultures (e.g., cells) into a cluster may be accelerated. The concave structure 2335 may be provided with reference to the concave structure in other embodiments of the present disclosure (e.g., embodiments of FIG. 14 and FIG. 15). For example, the concave structure 2335 may be provided with reference to the concave structure 1455, and the concave structure 2335 may comprise a porous membrane configured to retain a substance with a size larger than a pore size of the porous membrane. As another example, an inner wall of the concave structure 2335 may have cell adhesion inert (e.g., a membrane of inert material is provided on the inner wall of the concave structure 2335) to accelerate the aggregation of cells into a cluster within the concave structure 2335.

The through well 23321 may correspond to the culture inlet and outlet channel 2321. For example, the culture inlet and outlet channel 2321 includes a plurality of sample introduction wells disposed on a bottom wall of the sample introduction plate 2322, and the sample introduction wells are in one-to-one correspondence with the through wells 23321 to allow an operator to add a culture to the culture chambers 2331 through the culture inlet and outlet channel 2321.

In some embodiments, the culture fluid channel 2340 may include a culture fluid accommodation chamber. A detailed description of the culture fluid accommodation chamber can be found in other parts of the present disclosure (e.g., embodiments of FIGS. 28-30), and is not repeated here.

In some embodiments, one or more components of the culture device 2310 described above may be omitted. For example, the culture device 2310 may not include the sample introduction channel 2320, and the sample culture may be directly added to the culture chamber 2331. When a count of culture chambers 2331 exceeds one, the sample culture may be added well by well or utilizing a batch sample introduction tool (e.g., a row of needles).

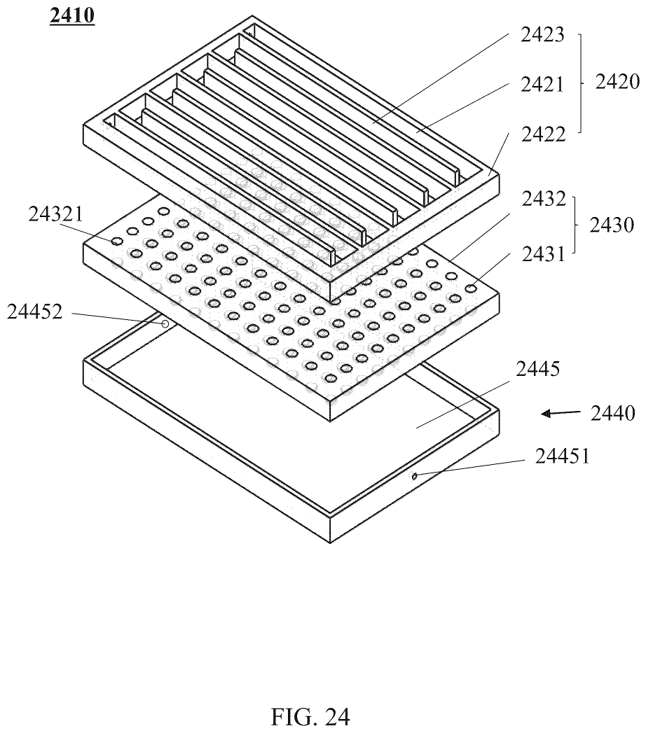

FIG. 24 is a schematic diagram illustrating an explosion structure of a culture device according to some embodiments of the present disclosure. As shown in FIG. 24, a culture device 2410 includes a sample introduction channel 2420, a chamber layer 2430, and a culture fluid channel 2440 that are independent of each other.

The sample introduction channel 2420 may include a sample introduction plate 2422 that includes a plurality of baffles 2423. A portion of the sample introduction plate 2422 that is not provided with the baffles forms a curved culture inlet and outlet channel(s) 2421. The culture inlet and outlet channel 2421 may be in flow communication with a plurality of culture chambers 2431 provided on the culture plate 2432.

In some embodiments, one or more components of culture device 2410 described above may be omitted. For example, the culture device 2410 may not include the sample introduction channel 2420, and a sample culture may be directly added to the culture chamber 2431. When a count of culture chambers 2431 exceeds one, the sample culture may be added well by well or utilizing a batch sample introduction tool (e.g., a row of needles).

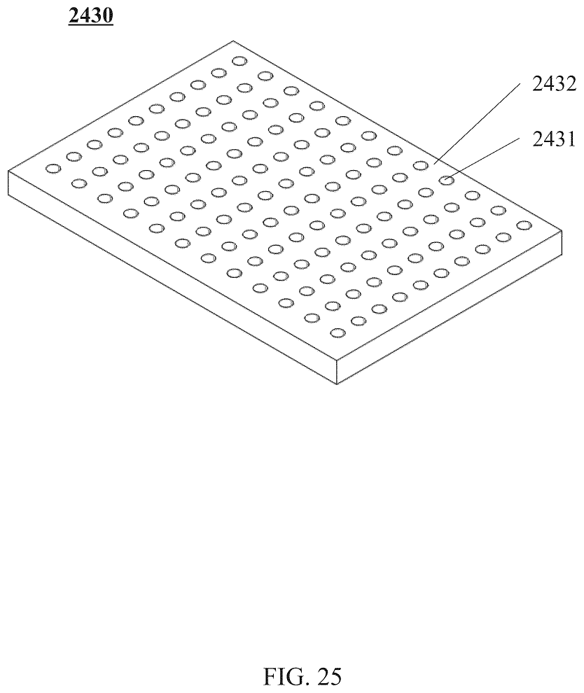

The chamber layer 2430 may include a culture plate 2432 and a culture chamber(s) 2431 provided on the culture plate 2432. For example, FIGS. 25-27 provide a detailed structural view of the chamber layer 2430. In particular, FIG. 25 is a schematic diagram illustrating an exemplary three-dimensional structure of the chamber layer 2430; FIG. 26 is a schematic diagram illustrating an exemplary side view of the chamber layer 2430; and FIG. 27 is a schematic diagram illustrating an exemplary cross-sectional view of the chamber layer along a direction A-A illustrated in FIG. 26. As shown in FIG. 26 and FIG. 27, a plurality of through wells 24321 are provided on the culture plate 2432, and a porous membrane 2460 is provided at a bottom of each through well 24321. The porous membrane 2460 may be in one-to-one correspondence with the through wells 24321 to form a plurality of culture chambers 2431.

In some embodiments, the porous membrane 2460 may be similar to porous membranes described in other portions of the present disclosure (e.g., the porous membrane 1660, the porous membrane 1860, etc.), which can allow only a specific substance (e.g., a culture fluid) to pass through, henceforth retaining other substances (e.g., a culture). In some specific embodiments, the porous membrane 2460 is in a shape of a hemisphere, such that when the porous membrane 2460 is mated to the through well 24321, a chamber layer 2431 with a downwardly concave bottom (i.e., protruding in a direction away from an interior of the through well 24321) may be formed. The culture chamber 2431 with a concave bottom is more suitable for a three-dimensional culture model. The culture chamber 2431 with a downwardly concave bottom may also be used in conjunction with a culture fluid accommodation chamber (e.g., the culture fluid accommodation chamber 2845 shown in FIG. 28) to update a culture fluid and/or other substances (e.g., a culture fluid to be screened) in the culture chamber 2431.

Continuing to refer to FIG. 24, the culture fluid channel 2440 may include a culture fluid accommodation chamber 2445, a culture fluid inlet 24451, and a culture fluid outlet 24452. The culture fluid accommodation chamber 2445 may be used to accommodate the culture fluid or other substances. The culture fluid inlet 24451 may be used to add the culture fluid or other substances to the culture fluid accommodation chamber 2445. The culture fluid outlet 24452 may be used to drain the culture fluid or other substances from the culture fluid accommodation chamber 2445.

When the sample introduction channel 2420, the chamber layer 2430, and the culture fluid channel 2440 are mated with each other, an exposed portion of the culture chamber 2431 outside the culture plate 2432 (i.e., a bottom formed by the porous membrane 2460) may be disposed, in whole or in part, in the culture fluid accommodation chamber 2445. When the culture fluid or other substances are added to the culture fluid accommodation chamber 2445, the culture fluid and/or the other substances may enter the culture chamber 2431 via the porous membrane 2460. The culture fluid and/or other substances in the culture fluid chamber 2431 may also be discharged into the culture fluid accommodation chamber 2445 via the porous membrane 2460 to allow the update and exchange of the culture fluid and/or other substances.

In the embodiment shown in FIG. 24, the culture fluid accommodation chamber 2445 includes only a single chamber used to perform a uniform fluid change and update of the culture chamber 2431. In some embodiments, the culture chamber 2431 may be divided into at least two sets of culture chambers, each of which may include one or more culture chambers 2431. In a controlled study, different culture fluids and/or other substances (e.g., drugs) may be added to a plurality of sets of culture chambers, and t the culture fluids and/or other substances in the plurality of sets of culture chambers may be updated separately. To improve the efficiency of adding or updating the sample culture fluid and/or other substances, a culture fluid accommodation chamber including a plurality of culture fluid sub-accommodation chambers is provided.

FIGS. 28 to 30 are schematic diagrams illustrating an exemplary culture fluid accommodation chamber comprising a plurality of culture fluid sub-accommodation chambers according to some embodiments of the present disclosure. As shown in FIG. 28, a culture fluid accommodation chamber 2845 may include at least one culture fluid sub-accommodation chamber 28453. Each culture fluid sub-accommodation chamber 28453 of the at least one culture fluid sub-accommodation chamber 28453 may correspond to one set of at least two sets of culture fluid chambers. Each culture fluid sub-accommodation chamber 28453 may be provided with an independent accommodation chamber inlet (not shown in the figures) and an accommodation chamber outlet (not shown in the figures). In this embodiment, when the plurality of culture fluid sub-accommodation chambers 28453 are provided, different culture fluids or other substances may be added to the plurality of culture fluid sub-accommodation chambers 28453 via the accommodation chamber inlets thereof. In this way, different culture fluids and/or other substances may be quickly added to each set of culture chambers to form desired control sets.

In the embodiment shown in FIG. 28, the culture fluid accommodation chamber 2845 may include 12 culture fluid sub-accommodation chambers 28453, forming a grid structure with three rows and four columns. Correspondingly, the culture chambers may also be divided into 12 sets. A count of culture chambers contained in each set of culture chambers may be any number. When the culture fluid accommodation chamber 2845 is placed below a culture plate (e.g., the culture plate 2432 shown in FIG. 24), exposed portions of each set of culture chambers outside the culture plate may be accommodated in a corresponding culture fluid sub-accommodation chamber 28453.

In the embodiment shown in FIG. 29, the culture fluid sub-accommodation chambers 29453 of the culture fluid accommodation chamber 2945 are also arranged in a grid structure. Different from FIG. 28, the culture fluid sub-accommodation chambers 29453 in the culture fluid accommodation chamber 2945 shown in FIG. 29 have a greater count of sets (e.g., 48 sets) and a smaller volume of the culture fluid sub-accommodation chambers 29453. In the embodiment shown in FIG. 30, the culture fluid accommodation chamber 3045 includes 3 culture fluid sub-accommodation chambers 30453, and the 3 culture fluid sub-accommodation chambers 30453 are arranged in a side-by-side channel structure.

In some embodiments, the plurality of culture fluid sub-accommodation chambers of the culture fluid accommodation chamber may be arranged in a form other than the grid structure or the side-by-side channel structure. In some embodiments, the plurality of culture fluid sub-accommodation chambers of the culture fluid accommodation chamber may be different in size. In some embodiments, the plurality of culture fluid sub-accommodation chambers may share an accommodation chamber inlet and/or an accommodation chamber outlet. Merely by way of example, culture fluid sub-accommodation chambers 28453 located in the same row in FIG. 28 may share an accommodation chamber inlet and an accommodation chamber outlet.

It is to be noted that shapes, sizes, positions, quantities, etc. of the culture device and its related components shown in FIGS. 23-30, and the related descriptions are for illustrative purposes only and do not limit the present disclosure to the scope of the embodiments cited. It will be understood that, for those skilled in the art, various deformations and modifications may be made without departing from the principle of the culture device and its related components after understanding this principle. For example, the sample introduction channel 2420 of FIG. 24 may be in other forms (e.g., similar to the sample introduction channel 220 shown in FIG. 2). As another example, the sample introduction plate 2422 may be provided with a plurality of culture inlet and outlet channels 2421, which may be used to add the sample culture to a plurality of sets of culture chambers 2431 respectively.

Beneficial effects of one or more embodiments of the present disclosure include, but are not limited to: (1) by providing a sample introduction channel for adding a culture to a chamber layer, and providing a culture fluid channel for updating a culture fluid or other substances in the culture chamber (for example, a drug to be screened), the impact on the culture in the culture chamber when adding or updating a sample culture fluid or other substances to the culture chamber may be avoided; (2) by connecting the sample introduction channel and/or the culture fluid channel with at least one culture chamber, a cumbersome process of adding a sample culture and/or updating the culture fluid well by well can be reduced, and the efficiency of the addition of the sample culture and/or update of the culture fluid can be improved; (3) by displacing the culture fluid channel and the sample introduction channel independently from the chamber layer, the manufacturing difficulty of the chamber layer can be reduced; (4) by providing a plurality of sets of culture fluid sub-channels or a plurality of culture fluid sub-accommodation chambers, the sample culture fluid may be added to a plurality of sets of culture chambers separately and be updated separately, and the efficiency of culture fluid addition and update can be improved; (5) by using a porous membrane as a sidewall and/or a bottom wall of the culture chamber, characteristics of the porous membrane can be used to improve the culture effect. For example, by utilizing the porous membrane with a retention function, the culture fluid or other substances can be updated without affecting the culture and the condition of a cell culture chamber; (6) by providing the culture chamber with an inert material (e.g., a membrane of inert material) in the culture chamber, the culture can be aggregated into a cluster more quickly, henceforth effectively improving a speed of a three-dimensional culture. It should be noted that the beneficial effects that may be produced by different embodiments are different, and the beneficial effects that may be produced in different embodiments may be any one or combination of any one or more of the above, or any other beneficial effect that may be obtained.

The basic concepts have been described above, and it is apparent to those skilled in the art that the foregoing detailed disclosure serves only as an example and does not constitute a limitation of the present disclosure. Although not explicitly stated here, those skilled in the art may make various modifications, improvements, and amendments to the present disclosure. These alterations, improvements, and modifications are intended to be suggested by this disclosure, and are within the spirit and scope of the exemplary embodiments of this disclosure.

Moreover, certain terminology has been used to describe embodiments of the present disclosure. As in “one embodiment”, “an embodiment”, and/or “some embodiments” means a feature, structure, or characteristic associated with at least one embodiment of the present disclosure. Accordingly, it should be emphasized and noted that two or more references to “an embodiment” or “one embodiment”,” or “an alternative embodiment” in different places in the present disclosure do not necessarily refer to the same embodiment. In addition, some features, structures, or features in the present disclosure of one or more embodiments may be appropriately combined.

In addition, unless expressly stated in the claims, the present disclosure does not deal with the order of elements and sequences, the use of numerical letters, or the use of other names, and is not intended to qualify the order in which one or more of the components of the present disclosure are assembled with one or more another. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure does not mean that the present disclosure object requires more features than the features mentioned in the claims. Rather, claimed subject matter may lie in less than all features of a single foregoing disclosed embodiment.

Finally, it should be understood that the embodiments in the present disclosure are used only to illustrate the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

1. A culture device, comprising:
a chamber layer including at least one culture chamber for accommodating a culture;
a sample introduction channel in flow communication with the at least one culture chamber, the sample introduction channel being configured to guide the culture into or out of the at least one culture chamber; and
a culture fluid channel in flow communication with the at least one culture chamber, the culture fluid channel being configured to guide a culture fluid into or out of the at least one culture chamber.

2. The culture device of claim 1, wherein at least one of the sample introduction channel or the culture fluid channel is disposed independently relative to the chamber layer.

3. The culture device of claim 1, wherein the sample introduction channel is disposed above the culture fluid channel.

4. The culture device of claim 1, wherein the sample introduction channel includes a sample introduction plate and a culture inlet and outlet channel, the culture inlet and the outlet channel being disposed on the sample introduction plate, and the culture inlet and outlet channel being in flow communication with the at least one culture chamber.

5. The culture device of claim 1, wherein the culture fluid channel includes:
a culture fluid inlet for inputting the culture fluid;
a culture fluid outlet for discharging the culture fluid; and
a culture fluid update channel configured to transport the culture fluid input via the culture fluid inlet to the at least one culture chamber.

6. The culture device of claim 5, wherein the at least two culture chambers include at least two sets of culture chambers, and each set of the at least two sets of culture chambers includes one or more culture chambers among the at least one culture chamber; and
the culture fluid channel includes at least two culture fluid sub-channels, each of the at least two culture fluid sub-channels corresponds to one set of the at least two sets of culture chambers, and each of the at least two culture fluid sub-channels includes a culture fluid sub-inlet, a culture fluid sub-outlet, and a culture fluid update sub-channel.

7. The culture device of claim 5, wherein the culture fluid outlet has a first connection with the at least one culture chamber, the culture fluid update channel has a second connection with the at least one culture chamber, and a cross-sectional dimension of at least one of the first connection and the second connection is smaller than a dimension of a constituent unit of the culture.

8. The culture device of claim 5, wherein the culture fluid outlet has a first connection with the at least one culture chamber;
the culture fluid update channel has a second connection with the at least one culture chamber; and
at least one of the first connection and the second connection is provided with a porous membrane for retaining the culture.

9. The culture device of claim 8, wherein a pore size of the porous membrane satisfies at least one of:
not larger than 5 micrometers,
in a range of 50 micrometers to 4 millimeters,
in a range of 1 nanometer to 100 nanometers,
in a range of 5 nanometers to 1 micron,
in a range of 100 nanometers to 10 micrometers, or
in a range of 10 micrometers to 1 centimeter.

10. (canceled)

11. The culture device of claim 5, wherein at least one of the culture fluid inlet, the culture fluid outlet, or the culture fluid update channel is integrally disposed on a surface of the chamber layer or within the chamber layer.

12. The culture device of claim 5, wherein the culture fluid outlet is located above the culture fluid update channel.

13. The culture device of claim 1, wherein the chamber layer comprises a culture plate, and the culture plate is provided with at least one through well; and
the culture device further comprises a sealing cover, wherein the sealing cover covers a bottom surface of the culture plate and forms the least one culture chamber with the at least one culture through well.

14. The culture device of claim 13, wherein the sealing cover has at least one concave structure;
the at least one culture through well and the at least one concave structure form the at least one culture chamber.

15. The culture device of claim 1, wherein the chamber layer comprises:
a culture plate;
at least one porous membrane disposed on the culture plate, wherein the at least one porous membrane forms the at least one culture chamber with the culture plate, and the at least one porous membrane is attached to or configured to form a side wall of the at least one culture chamber; and
a membrane of inert material disposed on an inner bottom wall of the at least one culture chamber.

16. (canceled)

17. The culture device of claim 15, wherein the culture device further comprises at least one rack disposed on the culture plate, each porous membrane of the at least one porous membrane is attached to a peripheral wall of one of the at least one rack, and the peripheral wall of the rack is provided with a skeletonized structure, the skeletonized structure being configured to enable circulation of the culture fluid.

18. (canceled)

19. (canceled)

20. The culture device of claim 1, wherein the chamber layer comprises:
a culture plate;
at least one through well disposed on the culture plate; and
a porous membrane disposed at a bottom end of each of the at least one through well, and the each through well forms the culture chamber with the porous membrane disposed at the bottom end of the through well.

21. The culture device of claim 20, wherein the culture fluid channel comprises a culture fluid accommodation chamber with an open end, and the culture fluid accommodation chamber is disposed below the culture plate.

22. The culture device of claim 21, wherein the at least one culture chamber comprises at least two sets of culture chambers, and each set of the at least two sets of culture chambers comprise one or more culture chambers of the at least one culture chamber; and
the culture fluid accommodation chamber comprises at least two culture fluid accommodation sub-channels, each of the at least two culture fluid accommodation sub-channels corresponds to one set of the at least two sets of culture chambers, and each of the at least two culture fluid accommodation sub-channels comprises an accommodation chamber inlet and an accommodation chamber outlet disposed independently.

23. (canceled)

24. The culture device of claim 20, wherein a material of the porous membrane includes an inert material or the inert material is attached to a surface of the porous membrane.

25. The culture device of claim 20, wherein the porous membrane forms a concave structure.

26-37. (canceled)
